(19) United States
(12) Patent Application Publication
(10) Pub. No.: US 2025/0164464 A1
(43) Pub. Date: May 22, 2025

(54) LIVER ORGANOID-DERIVED CULTURED HEPATOCYTES

(71) Applicant: OSAKA UNIVERSITY, Osaka (JP)

(72) Inventors: Hiroyuki MIZUGUCHI, Osaka (JP); Yukiko UEYAMA TOBA, Osaka (JP); Jumpei INUI, Osaka (JP)

(73) Assignee: OSAKA UNIVERSITY, Osaka (JP)

(21) Appl. No.: 18/840,088

(22) PCT Filed: Feb. 21, 2023
(86) PCT No.: PCT/JP2023/006121

§ 371 Date: Oct. 23, 2024

(30) Foreign Application Priority Data
Feb. 22, 2022 (JP) 2022-026065

(51) Int. Cl. G01N 33/50; C12N 5/071

(52) U.S. Cl. CPC G01N 33/5067; C12N 5/067; C12N 2501/11; C12N 2501/119; C12N 2501/12; C12N 2501/155; C12N 2506/45; C12N 2513/00

(57) ABSTRACT

Provided is a high-function hepatic organoid-derived cultured hepatocyte, the hepatocyte being applicable to the evaluation of pharmacokinetics or the like in drug discovery research or the like. Also provided is a method of culturing a hepatic organoid for the production of the high-function hepatocyte. Also provided is a high-function pluripotent stem cell-derived hepatic organoid or a method of producing the pluripotent stem cell-derived hepatic organoid. A high-function cultured hepatocyte in which the gene expression level of a drug-metabolizing enzyme is high can be produced by a method including a step of detaching a hepatic organoid from a substrate for an organoid to separate the organoid into a single cell, followed by its two-dimensional culture or spheroid culture. The hepatic organoid-derived cultured hepatocyte of the present invention maintains not only the gene expression of the drug-metabolizing enzyme but also high drug-metabolizing enzyme activity, and hence can be effectively utilized in an in vitro pharmacokinetics evaluation. In particular, the cultured hepatocyte does not use a substrate that has been required for the holding of a three-dimensional structure in a hepatic organoid, and hence the cultured hepatocyte can effectively evaluate the toxicity of a drug without capture of various test compounds in a substrate such as Matrigel.

### TECHNICAL FIELD

The present invention relates to a high-function cultured hepatocyte including a hepatic organoid-derived cell, the hepatocyte being utilizable in drug discovery research or the like, and to a hepatic organoid culture method for producing the high-function hepatocyte.

The present application claims priority from Japanese Patent Application No. 2022-026065, which is incorporated herein by reference.

### BACKGROUND ART

The prediction of the hepatopathy risk of a compound at an early stage in the research and development of a medicine is important for an increase in success rate of the research and development, and the curtailment of cost therefor and the period thereof. A liver is an organ that plays a central role in metabolism, and many therapeutic drugs are metabolized in the liver to show physiological activity and toxicity. A safety evaluation with a human hepatocyte is indispensable to drug discovery research because a drug-induced hepatopathy is a main cause for the suspension of the development of a medicine or the withdrawal thereof from the market. However, the number of human frozen hepatocytes, which have been generally used, to be supplied in one and the same lot is limited, and long-term culture thereof significantly reduces a hepatic function. Accordingly, it has been difficult to perform a large-scale and high-accuracy safety evaluation.

In recent years, an organoid culture technology has been attracting attention. An organoid is a three-dimensionally cultured (3D cultured) cell having a feature and proliferation potency similar to those of an organ three-dimensionally formed by utilizing a scaffold, such as a porous membrane or a hydrogel, and the research and development thereof have been vigorously performed in fields, such as regenerative medicine and tissue engineering, for creating each tissue or organ in a living body from the cell. Although two-dimensional culture (2D culture) has formerly been a general trend in a cell culture technology, three-dimensional culture to be performed in an environment close to the inside of the living body has currently started to become mainstream. It has been said that an organoid three-dimensionally formed by utilizing a scaffold has an excellent function close to that of the living body as compared to the two-dimensional culture.

A human hepatocyte obtains proliferation potency as a hepatic organoid when cultured under a specific culture condition. However, a human hepatic organoid has a problem in that its hepatic function is low, and hence its potential to find applications in drug discovery has not been investigated. Various investigations on a method of culturing a human hepatic organoid have been advanced (Non Patent Literatures 1 and 2). There is a report on a hepatic organoid (hepatobiliary tubular organoid: HBTO) in which a bile canaliculus and a bile duct formed by a hepatocyte are functionally connected to each other by the coculture of the hepatic progenitor cell and biliary epithelial cell of a mouse. Further, there is a report on the production of a HBTO having introduced thereinto a human hepatocyte, and the following has been reported (Non Patent Literature 3): a high albumin-secreting ability and high drug-metabolizing enzyme activity are maintained in the HBTO for a long time period, and hence it is revealed that bile acid or bilirubin taken in by a hepatocyte is transported from the hepatocyte to a bile duct as in a liver tissue in a living body.

However, each of the hepatic organoids is a three-dimensionally cultured cell to be cultured under the state of being embedded in a substrate for three-dimensional culture such as Matrigel (basement membrane matrix product). In the three-dimensional culture in a state in which the organoid is embedded in the substrate, concern is raised about the capture of various test compounds in the substrate, and hence an available experimental system is limited. The establishment of a technology for the culture of a high-function human hepatic organoid applicable to the safety evaluation of a drug has been desired.

### CITATION LIST

### Non Patent Literature

[NPL 1] Broutier et al., Nat Protoc., 2016 September; 11(9): 1724-43

[NPL 2] Hu et al., 2018, Cell 175, 1591-1606

[NPL 3] Tanimizu et al., NATURE COMMUNICATIONS, 2021, 12: 3390| https://doi.org/10.1038/s41467-021-23575-1

### SUMMARY OF INVENTION

### Technical Problem

An object of the present invention is to provide a high-function hepatic organoid-derived cultured hepatocyte, the hepatocyte being applicable to the evaluation of pharmacokinetics or the like in drug discovery research or the like. Another object of the present invention is to provide a method of culturing a hepatic organoid for the production of the high-function hepatocyte. Another object of the present invention is to provide a high-function pluripotent stem cell-derived hepatic organoid or a method of producing the pluripotent stem cell-derived hepatic organoid.

### Solution to Problem

In order to achieve the above-mentioned objects, the inventors of the present invention have made an attempt to establish a technology for the culture of a high-function hepatic organoid, and have made extensive investigations. As a result, the inventors have succeeded in differentiation and maturation into a high-function hepatocyte through the culture of a hepatic organoid-derived cell (hereinafter referred to as “hepatic organoid cell”) obtained by separating a hepatic organoid into a single cell. Thus, the inventors have obtained an excellent hepatocyte, which can evaluate drug-metabolizing enzyme activity and drug toxicity, and has a high general-purpose property, and have completed the present invention. Further, the inventors have obtained a high-function pluripotent stem cell-derived hepatic organoid through production from a cell obtained by culturing a pluripotent stem cell for at least 14 days or from an iPS-derived hepatocyte.

That is, the present invention includes the following.
1. A cultured hepatocyte, including a hepatic organoid-derived cell, wherein a gene expression level of a drug-metabolizing enzyme of the cultured hepatocyte is increased as compared to a gene expression level of the drug-metabolizing enzyme of a hepatic organoid.
2. The cultured hepatocyte according to the above-mentioned item 1, wherein the drug-metabolizing enzyme is one or a plurality of drug-metabolizing enzymes selected from CYP3A4,CYP1A2, CYP2B6, CYP2C9, CYP2C19, CYP2D6, CYP2E1, and UGT1A1.
3. The cultured hepatocyte according to the above-mentioned item 1 or 2, wherein a gene expression level of an adult hepatocyte marker in the cultured hepatocyte is comparable to or more than a gene expression level of the adult hepatocyte marker of the hepatic organoid.
4. The cultured hepatocyte according to the above-mentioned item 3, wherein the adult hepatocyte marker is one or a plurality of markers selected from albumin (ALB), hepatocyte nuclear factor 1-alpha (HNF1a), hepatocyte nuclear factor 1-alpha (HNF4a), and Na+-taurocholate co-transporting polypeptide (NTCP).
5. The cultured hepatocyte according to any one of the above-mentioned items 1 to 4, wherein the cultured hepatocyte is a two-dimensionally cultured hepatocyte obtained by two-dimensional culture or a spheroidized hepatocyte obtained by spheroid culture.
6. The cultured hepatocyte according to any one of the above-mentioned items 1 to 5, wherein the hepatic organoid is a liver cell-derived hepatic organoid or a pluripotent stem cell-derived hepatic organoid.
7. The cultured hepatocyte according to the above-mentioned item 6, wherein the pluripotent stem cell-derived hepatic organoid is an iPS cell-derived hepatic organoid.
8. A method of producing a cultured hepatocyte including a hepatic organoid-derived cell, the method including the following steps:
(1) a step of separating a hepatic organoid into a single cell; and
(2) a step of culturing a hepatic organoid cell separated as the single cell, the hepatic organoid cell being seeded on a substrate for two-dimensional culture, to produce a monolayer film.
9. A method of producing a cultured hepatocyte including a hepatic organoid-derived cell, the method including the following steps:
(1) a step of separating a hepatic organoid into a single cell; and
(2) a step of culturing a hepatic organoid cell separated as the single cell in an incubator for spheroid formation.
10. The method of producing a cultured hepatocyte according to the above-mentioned item 8 or 9, wherein in (2) the culturing step, the culturing is performed by using, as a culture solution, a medium containing one or two or more kinds of humoral factors selected from EGF, OsM, HGF, Dex, BMP4, BMP7, FGF7, FGF10, and FGF19.
11. The method of producing a cultured hepatocyte according to any one of the above-mentioned items 8 to 10, wherein in (2) the culturing step of the above-mentioned item 8 or 9, the culturing is performed by using, as a culture solution, a medium containing one or a plurality of kinds of inhibitors selected from a ROCK inhibitor, a TGF-β inhibitor, a MEK inhibitor, and a GSK-3 inhibitor.
12. The method of producing a cultured hepatocyte according to any one of the above-mentioned items 8 to 11, wherein in (2) the culturing step of the above-mentioned item 8 or 9, the culturing is performed by using, as a culture solution, a medium containing a MEK inhibitor, a TGF-β inhibitor, and a ROCK inhibitor.
13. The method of producing a cultured hepatocyte according to any one of the above- mentioned items 8 to 12, wherein the hepatic organoid is a liver cell-derived hepatic organoid or a pluripotent stem cell-derived hepatic organoid.
14. The method of producing a cultured hepatocyte according to the above-mentioned item 13, wherein the hepatic organoid is the pluripotent stem cell-derived hepatic organoid, and is a hepatic organoid produced from a cell obtained by culturing a pluripotent stem cell for at least 14 days.
15. The method of producing a cultured hepatocyte according to the above-mentioned item 13, wherein the hepatic organoid is the pluripotent cell-derived hepatic organoid, and is a hepatic organoid produced from an iPS-derived hepatocyte.
16. The method of producing a cultured hepatocyte according to any one of the above-mentioned items 8 to 15, wherein a medium to be used in production and/or culture of the hepatic organoid is a Hep-med medium or a Chol-med medium.
17. A cultured hepatocyte, which is produced by the production method of any one of the above-mentioned items 8 to 16.
18. A kit for evaluating pharmacokinetics and/or evaluating drug toxicity, including: the cultured hepatocyte of any one of the above-mentioned items 1 to 7 and 17; and a device and/or a reagent required for an inspection.
19. A method of evaluating pharmacokinetics and/or a method of evaluating drug toxicity, including using the cultured hepatocyte of any one of the above-mentioned items 1 to 7 and 17.
20. A medium for culturing a cultured hepatocyte including a hepatic organoid-derived cell, including: 1 μM to 50 μM of a ROCK inhibitor; and 0.1 μM to 5 μM of a TGF-β inhibitor.
21. A method of producing a pluripotent stem cell-derived hepatic organoid, including a step of producing the pluripotent stem cell-derived hepatic organoid from a cell obtained by culturing a pluripotent stem cell for at least 14 days.
22. A method of producing a pluripotent stem cell-derived hepatic organoid, comprising a step of producing the pluripotent stem cell-derived hepatic organoid from an iPS-derived hepatocyte.
23. A pluripotent stem cell-derived hepatic organoid, which is produced by the production method of the above-mentioned item 21 or 22.
24. A pluripotent stem cell-derived hepatic organoid, the pluripotent stem cell-derived hepatic organoid being intended for use in two-dimensional culture or spheroid culture.
25. The pluripotent stem cell-derived organoid according to the above-mentioned item 24, wherein the pluripotent stem cell-derived hepatic organoid is an iPS cell-derived hepatic organoid.

### Advantageous Effects of Invention

The hepatic organoid-derived cultured hepatocyte of the present invention (hereinafter referred to as “cultured hepatocyte of the present invention”) maintains not only the gene expression of the drug-metabolizing enzyme but also high drug-metabolizing enzyme activity. Accordingly, the cultured hepatocyte can be effectively utilized in an in vitro pharmacokinetics evaluation. Further, the cultured hepatocyte of the present invention shows excellent sensitivity to a hepatopathy-expressing drug. In particular, the cultured hepatocyte is extremely excellent in the following point: the cultured hepatocyte does not use a substrate serving as a scaffold for three-dimensional culture that has been required in a hepatic organoid, and hence the cultured hepatocyte can evaluate the toxicity of a drug without capture of various test compounds in a substrate such as Matrigel.

### BRIEF DESCRIPTION OF DRAWINGS

FIGS. 1 show effects on hepatocyte maturation when, in the monolayer culture of a hepatic organoid cell, the cell is cultured for 3 days by adding various humoral factors. FIG. 1A is an illustration of a culture protocol. FIG. 1B shows the results of the gene expression of albumin (ALB) under the respective conditions, and FIG. 1C shows the results of the gene expression of cytochrome P450 3A4 (CYP3A4) under the respective conditions (Example 1).

FIGS. 2 show effects on the hepatocyte maturation when, in the monolayer culture of the hepatic organoid cell, the cell is cultured for 3 days by adding various factors to be used at the time of differentiation induction. FIG. 2A shows the results of the gene expression of each of ALB and CYP3A4 at the time of the addition of the various factors, and shows that the addition of an epithelial-mesenchymal transition inhibitor (EMTi) containing a ROCK inhibitor, a MEK inhibitor, and a TGF-β inhibitor provides a particularly excellent result. FIG. 2B shows the results of the gene expression of ALB at the time of the addition of the EMTi and the various humoral factors used in Example 1, and FIG. 2C shows the results of the gene expression of CYP3A4 at the time of the addition of the EMTi and the various humoral factors used in Example 1 (Example 2).

FIGS. 3 show effects on the hepatocyte maturation when, in the monolayer culture of the hepatic organoid cell, the cell is cultured for 6 days by adding the EMTi and the various humoral factors. FIG. 3A is an illustration of a culture protocol. FIG. 3B shows the results of the gene expression of ALB at the time of the addition of the EMTi and the various humoral factors used in Example 1, and FIG. 3C shows the results of the gene expression of CYP3A4 at the time of the addition of the EMTi and the various humoral factors used in Example 1 (Example 3).

FIGS. 4 show effects on the hepatocyte maturation when, in the monolayer culture of the hepatic organoid cell, the cell is cultured for 9 days by adding the EMTi and the various humoral factors. FIG. 4A is an illustration of a culture protocol. FIG. 4B shows the results of the gene expression of ALB at the time of the addition of the EMTi and the various humoral factors used in Example 1, and FIG. 4C shows the results of the gene expression of CYP3A4 at the time of the addition of the EMTi and the various humoral factors used in Example 1. FIG. 4D is an illustration of a more specific culture protocol (Example 4).

FIGS. 5 show the results of the function evaluation of a cultured hepatocyte of the present invention. FIG. 5A is an illustration of a culture protocol, and FIG. 5B shows the activity of the cultured hepatocyte for a drug-metabolizing enzyme CYP3A4 (Example 5).

FIGS. 6 show the results of the function evaluation of the cultured hepatocyte of the present invention. FIG. 6A is an illustration of a culture protocol, and FIG. 6B shows the inducibilities of various drug-metabolizing enzymes (CYPB6, CYP1A2, and CYP3A4) (Example 6).

FIGS. 7 show the results of the function evaluation of the cultured hepatocyte of the present invention. FIG. 7A is an illustration of an experimental protocol, and FIG. 7B shows the influences of various hepatopathy-expressing drugs on a cell viability (Example 7).

FIGS. 8 show effects of the hepatocyte maturation of a spheroidized hepatocyte and a two-dimensionally cultured hepatocyte at the time of spheroid culture. FIG. 8A is an illustration of a culture protocol. FIG. 8B shows the results of the gene expression of each of ALB and CYP3A4 (Example 8).

FIGS. 9 show the results of a cell evaluation when the two-dimensionally cultured hepatocyte is cultured for a long time period of up to 15 days. FIG. 9A shows the results of the observation of the cell morphologies of the hepatocyte with a phase-contrast microscope. FIG. 9B shows the results of the analysis of various gene expression levels in a heatmap (Example 9).

FIGS. 10 show the results of a cell evaluation when the two-dimensionally cultured hepatocyte is cultured for a long time period of up to 30 days. FIG. 10A shows the results of the gene expression of each of ALB and CYP3A4. FIG. 10B shows the results of the measurement of CYP3A4 enzyme activity (Example 10).

FIGS. 11 show a hepatic organoid produced from human iPS cell-derived hepatocyte-like cells (HLCs). FIG. 11A is an illustration of a protocol for the production of a human hepatic organoid from an iPS cell, a protocol for maintenance culture, and the respective cell morphologies thereof. FIG. 11B shows the proliferation rate and cell morphology of the hepatic organoid produced from the human iPS cell-derived hepatocyte-like cells (HLCs). FIG. 11C shows the results of the measurement of the respective gene expression levels of the respective hepatocyte markers and drug-metabolizing enzymes in the hepatic organoid at each passage (Example 11).

FIGS. 12 show results obtained for a hepatocyte obtained by further two-dimensionally culturing the hepatic organoid produced from the human iPS cell-derived hepatocyte-like cells (HLCs). FIG. 12A is an illustration of a protocol for the production of the two-dimensionally cultured hepatocyte produced from the human iPS cell-derived hepatocyte-like cells (HLCs), and FIG. 12B shows cell morphologies in an EMTi-containing system and an EMTi-free system. FIG. 12C shows the results of the measurement of the respective gene expression levels of the respective hepatocyte markers and drug-metabolizing enzymes (Example 12).

FIG. 13 shows an influence of freezing on the hepatic organoid produced from the human iPS cell-derived hepatocyte-like cells (HLCs). FIG. 13 shows the results of the measurement of the respective gene expression levels of the respective hepatocyte markers and drug-metabolizing enzymes for the hepatic organoids at the time of frozen passage and at the time of unfrozen passage (Example 13).

FIGS. 14 show the results of the evaluations of the two-dimensionally cultured hepatocytes of hepatic organoid cells produced from the human iPS cell-derived hepatocyte-like cells (HLCs) under the respective conditions. FIG. 14A is an illustration of a protocol for the two-dimensional culture of the hepatic organoid produced from the iPS cell-derived hepatocyte-like cells, and FIG. 14B shows the results of the measurement of the respective gene expression levels of the respective hepatocyte markers and drug-metabolizing enzymes of the respective two-dimensionally cultured hepatocytes (Example 14).

FIGS. 15 show the results of the evaluations of the two-dimensionally cultured hepatocytes of hepatic organoid cells produced in the respective media. FIG. 15A is an illustration of a two-dimensional culture protocol, and FIG. 15B shows the results of the measurement of the respective gene expression levels of the respective hepatocyte markers and drug-metabolizing enzymes of the respective two-dimensionally cultured hepatocytes (Example 15).

FIGS. 16 show the results of the evaluations of two-dimensionally cultured hepatocytes cultured while medium conditions are changed. FIG. 16A is an illustration of a two-dimensional culture protocol, FIG. 16B shows the cell morphologies of the hepatocytes observed with a phase-contrast microscope, and FIG. 16C shows the results of the measurement of the respective gene expression levels of the hepatocyte markers and drug-metabolizing enzymes (Example 16).

FIGS. 17 show the results of the identification of the function of a hepatic organoid to be established by a difference in hepatic differentiation induction stage of a human iPS cell to be used. FIG. 17A shows a protocol for the production of the hepatic organoid from a cell at each stage of a hepatic differentiation induction process from the human iPS cell, and the cell morphology thereof. FIG. 17B shows the results of the analysis of various gene expression levels in a heatmap (Example 17).

FIGS. 18 show the results of the identification of the function of the hepatic organoid to be established by the difference in hepatic differentiation induction stage of the human iPS cell to be used. FIG. 18A is an illustration of a protocol for the production of the hepatic organoid from the cell at each stage of the hepatic differentiation induction process from the human iPS cell. FIG. 18B shows the results of the measurement of the respective gene expression levels of the respective hepatocyte markers and drug-metabolizing enzymes for the cells at the respective stages of the hepatic differentiation induction process and the respective hepatic organoids (Example 18).

FIG. 19 shows a protocol for the two-dimensional culture of a hepatic organoid produced from human iPS cell-derived hepatocyte-like cells (HLCs), and the cell morphology thereof observed with a phase-contrast microscope (Example 19).

FIGS. 20 show the results of the evaluations of the hepatic organoid produced from the human iPS cell-derived hepatocyte-like cells (HLCs) and a two-dimensionally cultured hepatocyte thereof. FIG. 20A is an illustration of a protocol for the two-dimensional culture of the hepatic organoid produced from the human iPS cell-derived hepatocyte-like cells, and FIG. 20B shows the results of the measurement of the respective gene expression levels of the respective hepatocyte markers and drug-metabolizing enzymes of the hepatic organoid. FIG. 20C shows the results of the measurement of the respective gene expression levels of the respective hepatocyte markers and drug-metabolizing enzymes of the two-dimensionally cultured hepatocyte (Example 20).

FIGS. 21 show the results of a cell evaluation when the two-dimensionally cultured hepatocyte is cultured for a long time period of up to 20 days. FIG. 21A is an illustration of a protocol for the long-term culture of the two-dimensionally cultured hepatocyte. FIG. 21B shows the cell morphologies of the hepatocyte observed with a phase-contrast microscope. FIG. 21C shows the results of the measurement of CYP3A4 enzyme activity (Example 21).

FIGS. 22 show the results of the function evaluation of the two-dimensionally cultured hepatocyte. FIG. 22A is an illustration of a culture protocol, and FIG. 22B shows the cell morphologies of the hepatocyte observed with a phase-contrast microscope (Example 22).

FIGS. 23 show an influence of cryopreservation on an iPS cell-derived hepatic organoid. FIG. 23A is an illustration of a protocol for the two-dimensional culture of the iPS cell-derived hepatic organoid after the cryopreservation. FIG. 23B shows the result of the measurement of CYP3A4 enzyme activity for each two-dimensionally cultured hepatocyte. FIG. 23C shows the results of the measurement of the respective gene expression levels of the hepatocyte markers and drug-metabolizing enzymes for hepatocytes obtained by two-dimensionally culturing the hepatic organoids at the time of frozen passage and at the time of unfrozen passage (Example 23).

FIG. 24 shows the result of the measurement of the cell proliferation potency of a hepatic organoid produced in each medium (Example 24).

FIGS. 25 show the results of the evaluations of hepatic organoids produced in the respective media. FIG. 25A is an illustration of a protocol for a hepatic organoid produced from human iPS cell-derived hepatocyte-like cells (HLCs). FIG. 25B shows the results of the measurement of the respective gene expression levels of the respective hepatocyte markers and drug-metabolizing enzymes for a hepatic organoid produced in a Hep-med medium. FIG. 25C shows the results of the measurement of the respective gene expression levels of the respective hepatocyte markers and drug-metabolizing enzymes for a hepatic organoid produced in a Chol-med medium (Example 25).

FIGS. 26 show results obtained for hepatocytes obtained by two-dimensionally culturing the hepatic organoids produced in the respective media. FIG. 26A is an illustration of a two-dimensional culture protocol. FIG. 26B shows the result of the measurement of the CYP3A4 enzyme activity of each two-dimensionally cultured hepatocyte. FIG. 26C shows the results of the measurement of the respective gene expression levels of the respective hepatocyte markers and drug-metabolizing enzymes of the respective two-dimensionally cultured hepatocytes (Example 26).

FIGS. 27 show results obtained for a hepatocyte obtained by two-dimensionally culturing a frozen organoid. FIG. 27A is an illustration of a protocol for the two-dimensional culture of an iPS cell-derived hepatic organoid. FIG. 27B shows the results of the measurement of the respective gene expression levels of the hepatocyte markers and drug-metabolizing enzymes for hepatocytes obtained by two-dimensionally culturing the hepatic organoid and the frozen hepatic organoid (Example 27).

### DESCRIPTION OF EMBODIMENTS

The present invention relates to a high-function cultured hepatocyte derived from a hepatic organoid, the hepatocyte being utilizable in drug discovery research or the like, and to a hepatic organoid culture method for producing the high-function hepatocyte. The present invention also relates to a high-function pluripotent stem cell-derived hepatic organoid or a method of producing the pluripotent stem cell-derived hepatic organoid.

### Hepatic Organoid-derived Cultured Hepatocyte

The cultured hepatocyte of the present invention is characterized in that the gene expression level of a drug-metabolizing enzyme is increased as compared to the gene expression level of the drug-metabolizing enzyme of a hepatic organoid. The phrase “increased as compared to the gene expression level of the drug-metabolizing enzyme of a hepatic organoid” means, for example, that when the gene expression level of the drug-metabolizing enzyme of the hepatic organoid is defined as 1, a gene is expressed at a level of 1.1 or more, preferably 1.5 or more, more preferably 3.0 or more, most preferably 5.0 or more.

Examples of the drug-metabolizing enzyme include one or a plurality of enzymes selected from a cytochrome P450 (CYP), a UDP-glucuronosyltransferase (UGT), an alcohol dehydrogenase, an aldehyde dehydrogenase, a glutathione peroxidase, a superoxide dismutase, a monoamine oxidase, a diamine oxidase, an epoxide hydrase, an esterase, an amidase, a glutathione S-transferase, a γ-glutamyl transpeptidase, an acetyltransferase, a sulfotransferase, and an enzyme involved in a drug transporter. Examples of the CYP include CYP3A4, CYP1A2, CYP2B6, CYP2C8, CYP2C9, CYP2C19, CYP2D6, and CYP2E1. Of those, CYP3A4, CYP1A2, CYP2B6, CYP2C9, CYP2C19, CYP2D6, and CYP2E1 are preferred, and CYP3A4 is most preferred. Examples of the UGT include UGT1A1, UGT1A3, UGT1A4, UGT1A6, UGT1A6, UGT1A9, UGT2B4, UGT2B7, UGT2B10, UGT2B11, and UGT2B15. Of those, UGT1A1, UGT1A6, UGT2B4, UGT2B7, and UGT2B15 are preferred, and UGT1A1 is most preferred.

Further, the cultured hepatocyte of the present invention is characterized in that the gene expression level of an adult hepatocyte marker is comparable to or more than the gene expression level of the adult hepatocyte marker of the hepatic organoid. Examples of the adult hepatocyte marker include albumin (ALB), hepatocyte nuclear factor 1-alpha (HNF1a), hepatocyte nuclear factor 1-alpha (HNF4a), and Na+-taurocholate co-transporting polypeptide (NTCP), and one or a plurality of markers are selected from these markers.

### Hepatic Organoid

The “hepatic organoid” as used herein only needs to be an organoid produced from a liver cell or a pluripotent stem cell-derived hepatocyte. Herein, the term “organoid” refers to a cluster (a cell population or a tissue construct) formed of organ-specific cells, and refers to a cultured cell having a feature and proliferation potency similar to those of an organ. The organoid is three-dimensionally formed by utilizing a scaffold substrate for cell culture, such as a porous membrane or a hydrogel. The scaffold substrate to be used in organoid culture (hereinafter referred to as “substrate for an organoid”) is used for accelerating the adhesion and proliferation of a cell to hold a three-dimensional structure, and substrates formed of various materials and having various pore sizes have been put into practical use. The substrate for an organoid of the present invention may be a substrate made of any material and having any structure, the substrate being known per se or to be developed in the future. The substrate is specifically, for example, a solubilized basement membrane extracted from EHS mouse sarcoma rich in an ECM protein containing a hydrogel, laminin (main component), type IV collagen, a heparin sulfate proteoglycan, entactin/nidogen, and various growth factors, and for example, a Matrigel (trademark) (Corning) basement membrane is used.

The hepatic organoid of the present invention is a liver cell-derived hepatic organoid or a pluripotent stem cell-derived hepatic organoid. Although the kind of the hepatic organoid in the present invention is not particularly limited, a human hepatic organoid is suitable. The hepatic organoid of the present invention may be cryopreserved with a cryopreservation medium. The cryopreservation of the organoid may be performed by a method known per se. The cryopreserved hepatic organoid may be thawed and reseeded. A method known per se or any method to be developed in the future may be applied as a method of thawing the cryopreserved organoid. The hepatic organoid of the present invention may be used in the cultured hepatocyte of the present invention, or a passaged hepatic organoid may be used.

The liver cell-derived hepatic organoid refers to a hepatic organoid produced from a fresh or cryopreserved liver tissue, and may be an organoid produced by a method known per se or any method to be developed in the future. The organoid may be produced, for example, through use of HepatiCult™ Organoid Growth Medium (Human) (STEMCELL Technologies). The organoid may be produced as follows: the fresh or cryopreserved liver tissue is turned into a single cell with, for example, a protease, such as trypsin, a collagenase, or Dispase I, EDTA, or EGTA; the cell is recovered and subjected to washing and centrifugation treatment; and then the organoid is produced from the cell by utilizing a substrate for an organoid. The density of seeded cells only needs to enable organoid formation, and is hence not particularly limited. However, the density is, for example, from 1×10 cells/40 μL droplet to 1×10⁷ cells/40 μL droplet, preferably from 1×10² cells/40 μL droplet to 1×10⁶ cells/40 μL droplet, most preferably about 1×10⁵ cells/40 μL droplet. The cells may be cultured with a medium for a hepatic organoid after, for example, their incubation at 37° C. for from 1 minute to 60 minutes, preferably from 1 minute to 30 minutes, more preferably about 15 minutes. For example, a medium described in Non Patent Literature 1 (sometimes referred to as “Chol-med medium” herein) or a medium described in Non Patent Literature 2 (sometimes referred to as “Hep-med medium” herein) may be used as the medium for a hepatic organoid. The medium may be appropriately replaced with a new one, and the replacement may be performed, for example, once per 2 to 3 days.

The pluripotent stem cell-derived hepatic organoid may be produced from, for example, induced pluripotent stem cells (iPS cells). Although an iPS cell-derived hepatic organoid may be produced from a cell in any differentiated state, that is, any one of an iPS-derived hepatic progenitor cell, an iPS-derived immature hepatocyte, and an iPS-derived hepatocyte, the organoid is most suitably produced from the iPS-derived hepatocyte. A method known per se or any method to be developed in the future may be applied as a method of inducing the differentiation of the iPS cell into the iPS-derived hepatic progenitor cell, the iPS-derived immature hepatocyte, or the iPS-derived hepatocyte. Specifically, a method described in WO 2011/052504 A1 or WO 2016/147975 A1 may be applied. The pluripotent stem cell-derived hepatic organoid may be produced from, for example, a cell obtained by culturing a pluripotent stem cell for at least 14 days, preferably 25 days. A method known per se or any method to be developed in the future may be applied as a method of producing an organoid from an iPS cell. The organoid may be produced, for example, as follows: an iPS-derived cell at each differentiation induction stage is detached from a substrate for iPS cell culture with, for example, a protease, such as trypsin, a collagenase, or Dispase I, EDTA, or EGTA to be turned into a single cell; the cell is recovered and subjected to washing and centrifugal separation treatment; and then the organoid is produced from the cell by utilizing a substrate for an organoid. A medium for a hepatic organoid, such as a Hep-med medium or a Chol-med medium, may be used as a medium to be used in the production of the iPS cell-derived hepatic organoid, and a Hep-med medium is preferred. The density of seeded cells only needs to enable organoid formation, and is hence not particularly limited. However, the density is, for example, from 1×10 cells/40 μL droplet to 1×10⁷ cells/40 μL droplet, preferably from 1×10² cells/40 μL droplet to 1×10⁶ cells/40 μL droplet, most preferably about 1×10⁵ cells/40 μL droplet. The cells may be cultured with a medium for a hepatic organoid, such as a Hep-med medium or a Chol-med medium, after, for example, their incubation at 37° C. for from 1 minute to 60 minutes, preferably from 1 minute to 30 minutes, more preferably about 15 minutes. Of those, a Hep-med medium is preferred. The medium may be appropriately replaced with a new one, and the replacement may be performed, for example, once per 2 to 3 days. One or two or more kinds of humoral factors selected from activin A, bone morphogenetic protein 4 (BMP4), fibroblast growth factor 4 (FGF4), hepatocyte growth factor (HGF), and oncostatin M (OsM) are suitably incorporated into the above-mentioned medium.

A medium that may be used in the maintenance and culture of the hepatic organoid only needs to enable the culture of a liver tissue-derived cell, and is hence not particularly limited. However, a medium containing, for example, a Minimum Essential Medium Eagle (MEM) or Roswell Park Memorial Institute (RPMI) medium as a main component, and containing, for example, Advanced™ DMEM/F12 (GIBCO) or a medium component described in Meritxell Huch et al., Nature vol. 494, p. 247-250 (2013) may be used as a main medium. Specifically, HepatiCult™ Organoid Growth Medium (Human) (STEMCELL Technologies) may be used. In initial culture, the above-mentioned medium component may be used while antibiotics, such as penicillin G sodium salt, streptomycin sulfate, and amphotericin B, for example, 1×Antibiotic-Antimycotic (Sigma-Aldrich), and a Rho-binding kinase inhibitor such as Y-27632 are each appropriately incorporated thereinto.

The hepatic organoid can be passaged irrespective of whether the organoid is a liver cell-derived hepatic organoid or a pluripotent stem cell-derived hepatic organoid. Although a split ratio is not particularly limited, the ratio may be set to, for example, from 1:1 to 1:10. A method obtained by improving an existing method such as a report by Miyoshi et al. (Miyoshi and Stappenbeck, Nat. Protoc. 8, 2471-2482, 2013) may be applied as a passage protocol. To passage the hepatic organoid, the following is performed: the organoid is suspended in a liquid containing at least any one of, for example, a protease, such as trypsin, a collagenase, or Dispase I, EDTA, and EGTA, for example, TrypLE Select™ (Thermo Fisher Scientific); the suspension is incubated at 37° C. so that its matrix may be degraded; pipetting, centrifugation, and the like are each performed a plurality of times, followed by the removal of a supernatant; and a pellet is resuspended (embedded) in the matrix to a concentration corresponding to the split ratio. The suspension is dropped into a culture substrate and solidified at 37° C., and then the medium is added to the culture substrate. Thus, the passage can be performed. After the passage, until, for example, the second day of culture, the above-mentioned medium component may be used while a Rho-binding kinase inhibitor such as Y-27632 is appropriately incorporated thereinto. In addition, throughout the entire period of the culture, the above-mentioned medium component may be used while antibiotics, such as penicillin G sodium salt, streptomycin sulfate, and amphotericin B, for example, 1×Antibiotic-Antimycotic (Sigma-Aldrich) are each appropriately incorporated thereinto.

### Method of Producing Hepatic Organoid-derived Cultured Hepatocyte

The cultured hepatocyte of the present invention having the above-mentioned properties is a two-dimensionally cultured hepatocyte obtained by two-dimensional culture or a spheroidized hepatocyte obtained by spheroid culture. The two-dimensionally cultured hepatocyte and the spheroidized hepatocyte in this description are each produced by using a hepatic organoid, which is formed by culture utilizing a substrate for an organoid, as a starting raw material through a step of separating the organoid into a single cell. A hepatic organoid-derived cell obtained by detaching the hepatic organoid from the substrate for an organoid to separate the organoid into a single cell is hereinafter also referred to as “hepatic organoid cell.”

A. Method of producing Two-dimensionally Cultured Hepatocyte

The two-dimensionally cultured hepatocyte out of the cultured hepatocytes of the present invention may be produced by a method including the following steps:
(1) a step of separating a hepatic organoid into a single cell; and
(2) a step of culturing a hepatic organoid cell separated as the single cell, the hepatic organoid cell being seeded on a substrate for two-dimensional culture, to produce a monolayer film.

A method of detaching the hepatic organoid from a substrate for an organoid to separate the organoid into the single cell only needs to be based on a method known per se, and is hence not particularly limited. However, the single cell may be separated by: incorporating the hepatic organoid into a liquid containing at least any one of, for example, a protease, such as trypsin, a collagenase, or Dispase I, EDTA, and EGTA, for example, TrypLE Select™; and subjecting the mixture to filtration, a centrifuge, pipetting, or the like.

The two-dimensionally cultured hepatocyte may be produced by culturing the hepatic organoid cell seeded on the substrate for two-dimensional culture to form the monolayer film. In this description, the culture in which the monolayer film is formed is referred to as “two-dimensional culture.” The substrate for two-dimensional culture is not particularly limited as long as a hepatocyte can be subjected to monolayer culture, and a substrate for culture known per se, such as a culture plate, a culture petri dish, or a culture flask, or any culture substrate to be developed in the future may be included in the category of the substrate. The culture substrate to be used in the two-dimensional culture is distinguished from a culture substrate to be used in three-dimensional culture to be used in organoid culture.

The seeding density of the hepatic organoid cells only needs to be the density at which the monolayer film can be formed after the culture, and hence the density is not particularly limited. However, the density may be set to, for example, from 1.0×10² cells to 5.0×10⁷ cells, preferably from 1.0×10⁴ cells to 5.0×10⁶ cells per 1 cm² of the culture substrate. An environment in which the culture is performed only needs to be an environment known per se, and is hence not particularly limited. In general, however, the cells may be two-dimensionally cultured under the conditions of 37° C.±1° C. and 5%±1% CO₂. Although the culture period of the two-dimensional culture is not particularly limited as long as the cells can survive, the two-dimensional culture may be performed for, for example, from 1 day to 60 days. A medium may be appropriately replaced with a new one in the middle of the culture, and subculture may be performed as required.

A HCM™ medium (LONZA), Dulbecco's Modified Eagle Medium (DMEM), DMEM/Nutrient Mixture F-12 (DMEM/F12), HepatoZYME (Gibco), and Willam's E (Gibco) serving as hepatocyte culture media may each be used as a culture solution to be used at the time of the production of the above-mentioned two-dimensionally cultured hepatocyte. Of those, a HCM™ medium (LONZA) may be preferably used. One or a plurality of kinds of inhibitors selected from a ROCK inhibitor, a TGF-β inhibitor, a MEK inhibitor, and a GSK-3 inhibitor are suitably added to the medium. Examples of the ROCK inhibitor include Y-27632, thiazovivin, and GSK429286. Examples of the TGF-β inhibitor include SB-431542, A83-01, and LDN193189. Examples of the MEK inhibitor include PD0325901, AZD6244, and BIX02189.

Examples of the GSK-3 inhibitor include CHIR99021 and 6-bromoindirubin-3-oxime (BIO). The ROCK inhibitor and the TGF-β inhibitor are particularly suitably added in combination to the medium. Further, the MEK inhibitor is suitably used in combination with the foregoing. It is suitable to add the ROCK inhibitor at from 1 μM to 50 μM, preferably from 5 μM to 30 μM, more preferably from 5 μM to 10 μM, and the TGF-β inhibitor at from 0.1 μM to 5 μM, preferably from 0.5 μM to 3 μM, more preferably from 1 μM to 3 μM, and the MEK inhibitor may be combined at from 0.01 μM to 1 μM, preferably from 0.1 μM to 1 μM, more preferably from 0.3 μM to 0.8 μM therewith. Specifically, the combination of Y-27632 serving as the ROCK inhibitor and SB-431542 serving as the TGF-β inhibitor is suitable. PD0325901 may be combined as the MEK inhibitor with the foregoing. The combination of Y-27632 (10 μM), PD0325901 (0.5 μM), and SB-431542 (2 μM) is sometimes referred to as “EMTi” in Examples below. In addition, throughout the entire period of the culture, the above-mentioned medium component may be used while antibiotics, such as penicillin G sodium salt, streptomycin sulfate, and amphotericin B, for example, 1×Antibiotic-Antimycotic (Sigma-Aldrich) are each appropriately incorporated thereinto.

It is suitable to incorporate one or two or more kinds of humoral factors selected from epidermal growth factor (EGF), oncostatin M (OsM), hepatocyte growth factor (HGF), dexamethasone (Dex), bone morphogenetic protein 4 (BMP4), bone morphogenetic protein 7 (BMP7), fibroblast growth factor 7 (FGF7), fibroblast growth factor 10 (FGF10), and fibroblast growth factor 19 (FGF19) into the above-mentioned medium. It is particularly suitable to incorporate Dex at from 1 μM to 100 μM, preferably from 1 μM to 10 μM, and FGF19 at from 10 ng/mL to 1,000 ng/mL, preferably from 10 ng/ml to 100 ng/mL.

B. Method of producing Spheroidized Hepatocyte

The spheroidized hepatocyte including the hepatic organoid-derived cell of the present invention may be produced by a method including the following steps:
(1) a step of separating a hepatic organoid into a single cell; and
(2) a step of culturing a hepatic organoid cell separated as the single cell in an incubator for spheroid formation.
   (1) Step of separating Hepatic Organoid into Single Cell

The hepatic organoid can be separated into the single cell by a method described in the step (1) in the method of producing a two-dimensionally cultured hepatocyte.

(2) Step of culturing Hepatic Organoid Cell separated as the Single Cell in Incubator for Spheroid Formation

A spheroid may be produced by seeding the hepatic organoid cell on the incubator for spheroid formation. The term “spheroid” as used herein refers to the following cell cluster: the cluster does not require a scaffold substrate for cell culture for holding a three-dimensional structure, and cells adhere to each other under a floating state in a culture container to form the cluster. The “spheroidized cell” of the present invention refers to a cell cluster formed of a three-dimensional structure based on a spheroid produced from a liver cell or a pluripotent stem cell-derived hepatocyte. An incubator known per se or an incubator to be developed in the future may be used as the incubator for spheroid formation. For example, an incubator subjected to cell low-adsorption treatment, such as a culture flask or petri dish, a 96-well culture plate, a 384-well culture plate, or a 400-well culture plate, may be used, and an incubator whose well shape is, for example, a U-bottom, V-bottom, or spindle bottom shape may be used. Specifically, for example, Nunclon Sphera 96U Bottom Plate (manufactured by Thermo Fisher Scientific), EZSPHERE SP MICROPLATE 24 Well with Lid (model number: 4820-900SP) (manufactured by IWAKI Glass Co., Ltd.), Elplasia plate with Lid 24-Well Round Bottom 500/400, Ultra-Low Attachment (model number: 4441) (manufactured by Corning), and SPHERICAL PLATE 5D, 3D cell culture rEvolution (manufactured by Mitokogyo Corporation) may be used.

As in the culture solution to be used at the time of the production of the above-mentioned two-dimensionally cultured hepatocyte, a HCM™ medium (LONZA), Dulbecco's Modified Eagle Medium (DMEM), DMEM/Nutrient Mixture F-12 (DMEM/F12), HepatoZYME (Gibco), and Willam's E (Gibco) serving as hepatocyte culture media may each be used as a culture solution to be used at the time of the production of the above-mentioned spheroidized hepatocyte. Of those, a HCM™ medium (LONZA) may be preferably used. With regard to an additive or the like, an additive or the like to be used at the time of the production of the above-mentioned two-dimensionally cultured hepatocyte may be used.

### Methods of maintaining and storing Hepatic Organoid-derived Hepatocyte

The two-dimensionally cultured hepatocyte of the present invention may be cultured for a long time period of 15 or more days, preferably 20 or more days, more preferably 30 or more days. As in the culture solution to be used at the time of the production of the above-mentioned two-dimensionally cultured hepatocyte, a HCM™ medium (LONZA), Dulbecco's Modified Eagle Medium (DMEM), DMEM/Nutrient Mixture F-12 (DMEM/F12), HepatoZYME (Gibco), and Willam's E (Gibco) serving as hepatocyte culture media may each be used as a culture solution for maintenance to be used in the long-term culture. The combination of a ROCK inhibitor, a MEK inhibitor, and a TGF-β inhibitor is particularly suitably added to the medium. Specifically, it is more suitable to add the ROCK inhibitor at from 1 μM to 50 μM, preferably from 5 μM to 30 μM, more preferably from 5 μM to 10 μM, the MEK inhibitor at from 0.01 μM to 1 μM, preferably from 0.1 μM to 1 μM, more preferably from 0.3 μM to 0.8 μM, and the TGF-β inhibitor at from 0.1 μM to 5 μM, preferably from 0.5 μM to 3 μM, more preferably from 1 μM to 3 μM. It is suitable to further incorporate one or two or more kinds of humoral factors selected from EGF, OsM, HGF, Dex, BMP4, BMP7, FGF7, FGF10, and FGF19 into the above-mentioned medium. Dex (from 1 μM to 100 μM) and FGF19 (from 10 ng/mL to 1,000 ng/mL) are particularly suitably incorporated. The maintenance medium may be appropriately replaced with a new one. Although the frequency at which the medium is replaced with a new one is not particularly limited, the replacement may be performed, for example, every 1 to 15 days, and the replacement may be performed suitably every 1 to 7 days, more suitably every 1 to 3 days. Throughout the entire period of the culture, the above-mentioned medium component may be used while antibiotics, such as penicillin G sodium salt, streptomycin sulfate, and amphotericin B, for example, 1×Antibiotic-Antimycotic (Sigma-Aldrich) are each appropriately incorporated thereinto.

The two-dimensionally cultured hepatocyte or the spheroidized hepatocyte may be appropriately subcultured, and may be cryopreserved with a medium for cryopreservation. A method known per se or any method to be developed in the future may be applied as each of methods for the subculture and the cryopreservation of the cell.

### Utilization of Hepatic Organoid-derived Cultured Hepatocyte

As described above, in the cultured hepatocyte of the present invention, the gene expression level of the drug-metabolizing enzyme is increased as compared to the gene expression level of the drug-metabolizing enzyme of the hepatic organoid, and for example, when the gene expression level of the drug-metabolizing enzyme of the hepatic organoid is defined as 1, the gene is expressed at a level of 1.1 or more, preferably 1.5 or more, more preferably 3.0 or more, most preferably 5.0 or more. In addition, the cultured hepatocyte of the present invention maintains not only the gene expression of the drug-metabolizing enzyme but also high activity for the drug-metabolizing enzyme. Accordingly, the cultured hepatocyte can be effectively utilized in an in vitro pharmacokinetics evaluation. Further, the cultured hepatocyte of the present invention shows excellent sensitivity to a hepatopathy-expressing drug. In particular, the cultured hepatocyte is extremely excellent in the following point: the cultured hepatocyte does not use a substrate serving as a scaffold for three-dimensional culture, and hence can evaluate the toxicity of a drug without capture of various test compounds in a substrate such as Matrigel. According to the method of producing a cultured hepatocyte of the present invention, a large amount of cells each having such excellent performance can be supplied in one and the same lot, and hence hepatocytes each having certain quality can be supplied semipermanently. Such hepatocytes each having excellent quality or a cell population thereof may be used in a kit for evaluating pharmacokinetics or evaluating drug toxicity. Thus, the hepatopathy risk of a compound can be predicted at an early stage in the research and development of a medicine such as drug discovery and development, and the prediction is extremely useful in increasing the success rate of the research and development, and curtailing cost therefor and the period thereof. The present invention also covers a kit for evaluating pharmacokinetics and/or evaluating drug toxicity, the kit including: the cultured hepatocyte of the present invention; and a device and/or a reagent required for a pharmacokinetic evaluation or a drug toxicity evaluation. Further, the present invention covers a method of evaluating pharmacokinetics and/or a method of evaluating drug toxicity, the method including using the cultured hepatocyte of the present invention.

Further, the cultured hepatocyte of the present invention may be used in regenerative medicine or cell therapy in, for example, a case in which liver transplantation has heretofore been required. The regenerative medicine based on the cultured hepatocyte of the present invention may be capable of repairing a damaged hepatocyte and returning the function of a fibrillated liver to a normal one for a patient having a disease, such as hepatitis, a fatty liver, autoimmune hepatitis, or a liver cancer, and hence an effective therapeutic effect can be expected.

### EXAMPLES

The present invention is specifically described below by way of Examples for better understanding of the present invention. Needless to say, however, Examples are not intended to limit the scope of the present invention.

### (Example 1) Screening of Monolayer Culture Conditions (Zeroth Day to Third Day of Culture)

In this Example, culture conditions for the monolayer culture of a hepatic organoid produced from a commercial human frozen hepatocyte (XenoTech) were investigated. TrypLE Select™ was caused to act on the human frozen hepatocyte to detach the hepatocyte, and the resultant single cells were recovered and subjected to washing and centrifugal separation treatment, followed by their embedment in Matrigel™ (Corning) (estimated value of a seeding density=5×10⁵ cells/40 μL droplet). After having been incubated at 37° C. for 15 minutes, the cells were cultured with a medium for a hepatic organoid (Chol-med medium) to produce hepatic organoids.

The hepatic organoids were removed from the Matrigel™, and were seeded at 3.0×10⁵ cells/well on a collagen-coated 48-well plate, followed by their two-dimensional culture for 72 hours. The hepatic organoids removed from the Matrigel™ are also simply referred to as “hepatic organoid cells” in this Example and Examples below. The hepatic organoid cells were cultured with media obtained by further adding various humoral factors to a hepatocyte culture medium (HCM™ medium, LONZA) serving as a basic medium, and screening concerning their differentiation and maturation into liver cells was performed.

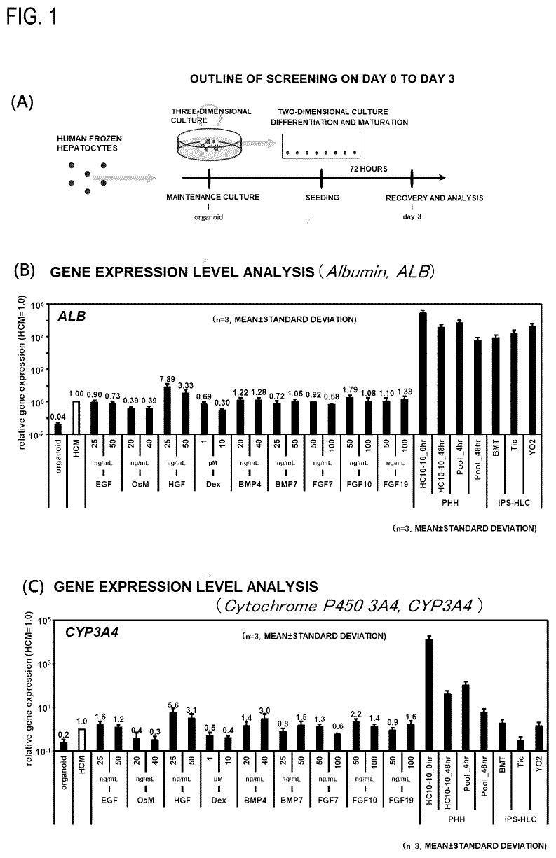

The hepatic organoid cells were cultured while each of recombinant proteins EGF, OsM, HGF, Dex, BMP4, BMP7, FGF7, FGF10, and FGF19 was added as a humoral factor at each of two concentrations, that is, a high concentration and a low concentration to the HCM™ medium. After having been two-dimensionally cultured for 72 hours, the cells were recovered, and the respective gene expression levels of albumin (ALB) and CYP3A4 serving as liver cell markers were analyzed (FIG. 1A).

As a comparative example, the gene expression levels of primary human hepatocytes (PHHs) (XenoTech) and human iPS cell-derived hepatocyte-like cells (iPS-HLCs) were also identified. The iPS-HLCs were produced by a method described in WO 2011/052504 A1. With regard to the PHHs, frozen cells were thawed, and were then cultured with the HCM™ medium for 4 hours and 48 hours, followed by the recovery of the cells. A mRNA extract immediately after the freezing and thawing was referred to as “HC10-10_0hr”, and mRNA extracts after the culture for the respective time periods were referred to as “HC10-10_4hr” and “HC10-10_48 hr”. The term “HC10-10” represents a lot number. The term “Pool” represents the pool sample of the PHHs having a lot number starting from FCL, OHO, or YOW. The terms “BMT”, “Tic”, and “YO2” in the iPS-HLCs each represent the strain name of human iPS cells.

The respective gene expression levels of ALB and CYP3A4 were measured by a quantitative RT-PCR method for the respective cells after their culture. A gene expression level when the hepatic organoid cells were cultured only with the HCM™ medium was defined as 1 (HCM=1.0). The cells were cultured with the HCM™ medium having added thereto each of the above-mentioned nine kinds of humoral factors at each of the two concentrations, that is, the high concentration and the low concentration. As a result, no differences were found in the respective gene expression levels of ALB and CYP3A4 (FIG. 1B and FIG. 1C).

### (Example 2) Screening of Monolayer Culture Conditions (Third Day of Culture)

Culture conditions at the time of the monolayer culture of hepatic organoid cells when a HCM™ medium (LONZA) was used as a basic medium in the same manner as in Example 1 and various factors were added thereto were investigated.

### (1) Addition of Various Compounds

The HCM™ medium (LONZA) was used as a basic medium in the same manner as in Example 1, and a glycogen synthase kinase 3 (GSK-3) inhibitor, a transforming growth factor-β (TGF-β) inhibitor, and an epithelial-mesenchymal transition (EMT) inhibitor to be used at the time of differentiation induction were each added thereto under the following various conditions, followed by screening concerning differentiation and maturation at the time of the monolayer culture of the hepatic organoid cells. The respective gene expression levels of ALB and CYP3A4 were analyzed by a quantitative RT-PCR method through the same approach as that of Example 1. A Rho-associated coiled-coil forming kinase (ROCK) inhibitor, a mitogen-activated extracellular signal-regulated kinase (MEK) inhibitor, and the TGF-β inhibitor were each used as the EMT inhibitor.
GSK-3 inhibitor: CHIR99021 (3 μM), 6-bromoindirubin-3-oxime (BIO, 5 μM)
TGF-β inhibitor: A83-01 (5 μM), LDN193189 (300 nM)
EMT inhibitor combination (EMTi): Y27632 (ROCK inhibitor, 10 μM)+PD0325901 (MEK inhibitor, 0.5 M)+SB431542 (TGF-β inhibitor, 2 μM)

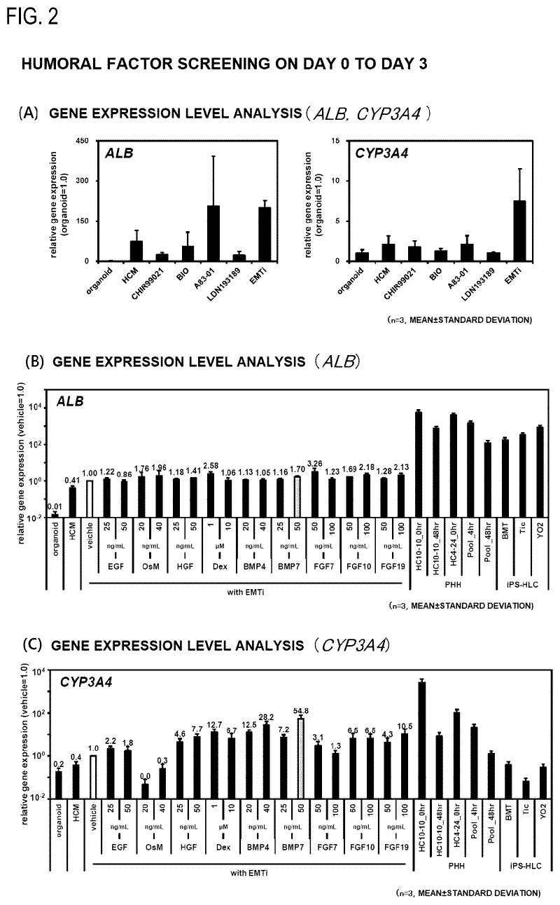

As a result, a significant increase in gene expression level of each of ALB and CYP3A4 by the addition of the EMTi was observed (FIG. 2A).

### (2) Addition of Various Humoral Factors (Recombinant Proteins)

The hepatic organoid cells were cultured with the HCM™ medium having added thereto the above-mentioned EMTi and having added thereto EGF, OsM, HGF, Dex, BMP4, BMP7, FGF7, FGF10, or FGF19 at each of two concentrations, that is, a high concentration and a low concentration in the same manner as in Example 1. The respective gene expression levels of ALB and CYP3A4 serving as liver cell markers after 72 hours of two-dimensional culture were analyzed by a quantitative RT-PCR method in the same manner as in Example 1. As a comparative example, the gene expression levels of PHHs cultured only with the HCM™ medium and iPS-HLCs were also identified in the same manner as in Example 1.

The respective gene expression levels of ALB and CYP3A4 were measured by a quantitative RT-PCR method for the respective cells after their culture. A gene expression level when the hepatic organoid cells were cultured only with a medium obtained by adding the above- mentioned EMTi to the HCM™ medium was defined as 1 (vehicle=1.0). As a result, no difference in gene expression level of ALB was found between the medium containing only the EMTi and the media containing the respective humoral factors (FIG. 2B). In contrast, the gene expression level of CYP3A4 showed an increasing tendency, and particularly in the combined use of the EMTi and BMP7 (50 ng/mL), the gene expression level of CYP3A4 increased by a factor of 50 (FIG. 2C).

### (Example 3) Screening of Monolayer Culture Conditions (Sixth Day of Culture)

Culture conditions up to the sixth day of culture when hepatic organoid cells were subjected to monolayer culture with a HCM™ medium having added thereto each of various factors were investigated in the same manner as in each of Examples 1 and 2.

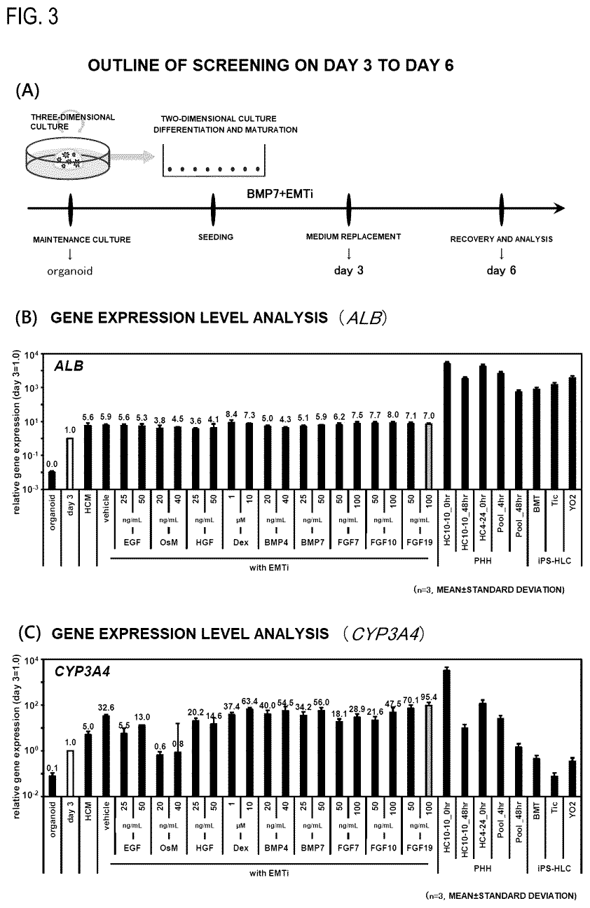

The hepatic organoid cells were seeded at 3.0×10⁵ cells/well on a collagen-coated 48-well plate, and were differentiated with the HCM™ medium having added thereto the EMTi and BMP7 (50 ng/mL) described in Example 2 until the third day of the culture. On the third day of the culture, the cells were cultured with the HCM™ medium having added thereto the above-mentioned EMTi and having added thereto EGF, OsM, HGF, Dex, BMP4, BMP7, FGF7, FGF10, or FGF19 at each of two concentrations, that is, a high concentration and a low concentration in the same manner as in Example 1 (FIG. 3A). The respective gene expression levels of ALB and CYP3A4 serving as liver cell markers after 6 days of two-dimensional culture were analyzed by a quantitative RT-PCR method. As a comparative example, the gene expression levels of PHHs cultured only with the HCM™ medium and iPS-HLCs were also identified in the same manner as in Example 1.

The respective gene expression levels of ALB and CYP3A4 were measured by a quantitative RT-PCR method for the respective cells after their culture. A gene expression level when the hepatic organoid cells were cultured only with the HCM™ medium having added thereto the above-mentioned EMTi was defined as a vehicle. Comparison was performed while the gene expression level of ALB of the cells cultured by using the EMTi and BMP7 (50 ng/mL) in combination on the third day of the culture was defined as 1.0. As a result, the respective gene expression levels of ALB and CYP3A4 showed increasing tendencies as compared to those on the third day of the culture, but no increases in gene expression level of ALB by the addition of the respective humoral factors were observed (FIG. 3B). In contrast, in the combined use of the EMTi and each of the various humoral factors, the gene expression level of CYP3A4 showed an increasing tendency, and in particular, the gene expression levels of CYP3A4 in the group on which the EMTi and BMP7 (50 ng/ml) had been caused to act, and the group on which the EMTi and FGF19 (100 ng/ml) had been caused to act increased by factors of about 50 and about 95, respectively (FIG. 3C).

### (Example 4) Screening of Monolayer Culture Conditions (Ninth Day of Culture)

Culture conditions up to the ninth day of culture when hepatic organoid cells were subjected to monolayer culture while each of various factors was added were investigated in the same manner as in each of Examples 1 to 3.

The hepatic organoid cells were seeded at 3.0×10⁵ cells/well on a collagen-coated 48-well plate, and were differentiated with the HCM™ medium having added thereto the EMTi and BMP7 (50 ng/mL) described in Example 2 until the third day of the culture. The medium was replaced with a new one on the third day of the culture, and the cells were differentiated with the HCM™ medium having added thereto the EMTi and FGF19 (100 ng/mL) until the sixth day of the culture.

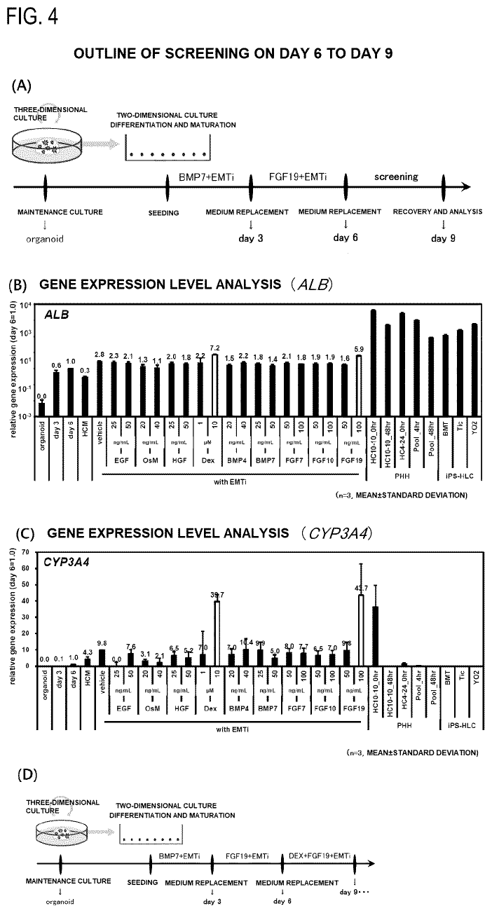

On the sixth day of the culture, the cells were cultured with the HCM™ medium having added thereto the above-mentioned EMTi and having added thereto EGF, OsM, HGF, Dex, BMP4, BMP7, FGF7, FGF10, or FGF19 at each of two concentrations, that is, a high concentration and a low concentration in the same manner as in Example 1 (FIG. 4A). The respective gene expression levels of ALB and CYP3A4 serving as liver cell markers after 9 days of two-dimensional culture were analyzed by a quantitative RT-PCR method. As a comparative example, the gene expression levels of PHHs cultured only with the HCM™ medium and iPS-HLCs were also identified in the same manner as in Example 1.

The respective gene expression levels of ALB and CYP3A4 were measured by a quantitative RT-PCR method for the respective cells after their culture. A gene expression level when the hepatic organoid cells were cultured only with the HCM™ medium having added thereto the above-mentioned EMTi was defined as a vehicle. Comparison was performed while the gene expression level of ALB of the cells cultured with the HCM™ medium having added thereto the EMTi and FGF19 (100 ng/mL) on the sixth day of the culture was defined as 1.0. The gene expression level of ALB on the third day of the culture was 0.6. As a result, the gene expression level of ALB showed an increasing tendency in each group as compared to that on the sixth day of the culture. Although the gene expression level of ALB showed an increasing tendency particularly in each of the group on which the EMTi and Dex (10 mM) had been caused to act, and the group on which the EMTi and FGF19 (100 ng/mL) had been caused to act, no significant difference was found between the respective groups (FIG. 4B). The gene expression level of CYP3A4 also showed an increasing tendency in each group as compared to that on the sixth day of the culture. In particular, the gene expression level of CYP3A4 increased by a factor of about 40 in each of the group on which the EMTi and Dex (10 μM) had been caused to act, and the group on which the EMTi and FGF19 (100 ng/mL) had been caused to act (FIG. 4C).

As can be seen from the foregoing, when the hepatic organoid cells are subjected to monolayer culture, the hepatic organoid cells can be effectively differentiated and matured by: culturing the cells with the HCM™ medium having added thereto the EMTi and BMP7 (50 ng/ml) described in Example 2 until the third day; culturing the cells with the HCM™ medium having added thereto the EMTi and FGF19 (100 ng/ml) on the third day of the culture until the sixth day; and culturing the cells with the HCM™ medium having added thereto the EMTi, FGF19 (100 ng/ml), and Dex (10 μM) on the sixth day of the culture (FIG. 4D). Two-dimensionally cultured hepatocytes produced by the method illustrated in FIG. 4D are also referred to as “hepatocytes (2D differentiated cells) of the present invention” in this Example and Examples below.

### (Example 5) Hepatocyte Function Evaluation (Activity for Drug-metabolizing Enzyme CYP3A4)

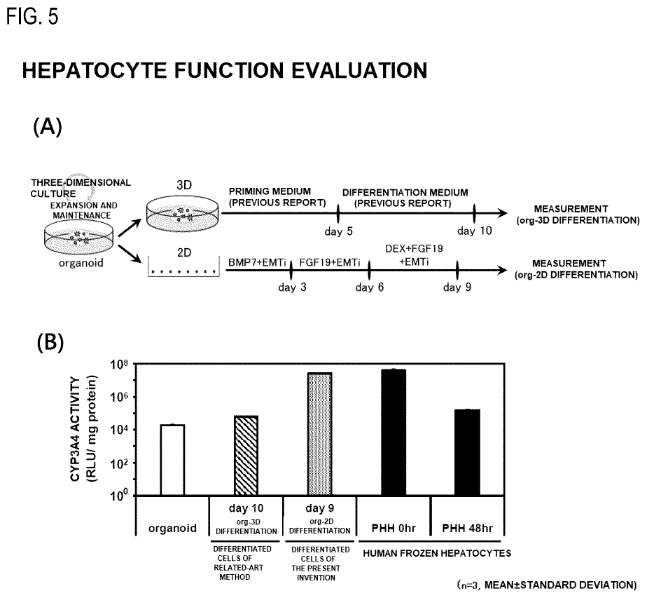

In this Example, hepatic organoid cells, three-dimensionally cultured cells produced by a method described in Non Patent Literature 1 (hereinafter also referred to as “hepatocytes (3D differentiated cells) of the related-art method”), and the hepatocytes (2D differentiated cells) of the present invention were evaluated for their functions by using activity for the drug-metabolizing enzyme CYP3A4 as an indicator (FIG. 5A). As a comparative example, with regard to primary human hepatocytes (PHHs), frozen cells were thawed, and were then cultured with a HCM™ medium for 48 hours. A culture supernatant immediately after the freezing and thawing was referred to as “PHH-Ohr”, and a culture supernatant after the 48 hours of culture was referred to as “PHH-48 hr”. The CYP3A4 activity was measured with P450-Glo™ CYP3A4 Assay and Screening System (Promega). Luciferin-IPA was used as a substrate for CYP3A4, and its light emission was measured with a luminometer (Lumat LB 9507, Berthold). The CYP3A4 activity was corrected with a protein mass in each well.

It was recognized that the hepatocytes (2D differentiated cells) of the present invention had CYP3A4 activity higher than that of the hepatocytes (3D differentiated cells) of the related-art method, and the activity was comparable to that of the primary human hepatocytes (PHHs) immediately after the freezing and thawing (FIG. 5B).

### (Example 6) Hepatocyte Function Evaluation (Induction of Drug-metabolizing Enzyme)

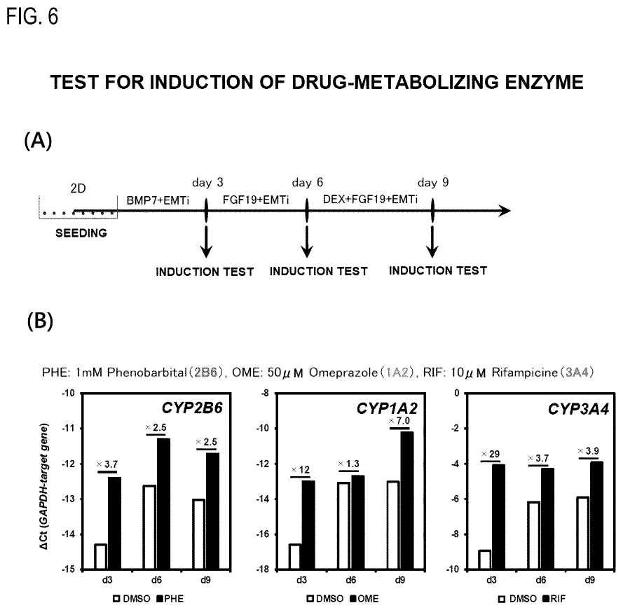

In this Example, the hepatocytes (2D differentiated cells) of the present invention were evaluated for the induction of a drug-metabolizing enzyme on the third day, sixth day, and ninth day of their culture (FIG. 6A). On the third day, sixth day, or ninth day of the culture, phenobarbital (PHE, 1 mM) serving as a CYP2B6 inducer, omeprazole (OME, 50 μM) serving as a CYP1A2 inducer, or rifampicin (RIF, 10 μM) serving as a CYP3A4 inducer was added to a medium, and was caused to act on the hepatocytes for 24 hours. After that, the ΔCt value ((Ct value of GAPDH)-(Ct value of target gene)) of each CYP gene was measured for each cell. As a control, dimethylsulfoxide (DMSO) serving as a solvent for each metabolizing enzyme activator was added thereto. The hepatocytes (2D differentiated cells) of the present invention had high sensitivity to each of the various drug-metabolizing enzyme inducers, and hence the gene expression levels of the respective drug-metabolizing enzymes were further enhanced by the respective inducers (FIG. 6B).

### (Example 7) Hepatocyte Function Evaluation (Evaluation of Toxicity exhibited by Hepatopathy-expressing Drug)

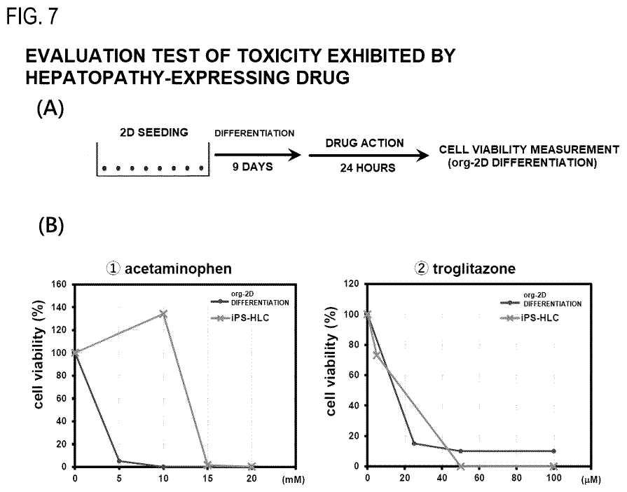

In this Example, the hepatocytes (2D differentiated cells) of the present invention were evaluated for toxicity exhibited by a hepatopathy-expressing drug on the ninth day of their culture. On the ninth day of the culture, acetaminophen or troglitazone known to express a hepatopathy was caused to act at each concentration on the hepatocytes for 24 hours, and then a cell viability was measured by a WST8 assay (FIG. 7A). As a control, the viability of human iPS cell-derived hepatocyte-like cells was similarly measured. It was suggested that the hepatocytes of the present invention were applicable to a hepatotoxicity evaluation test.

### (Example 8) Function Evaluation by Spheroid Culture

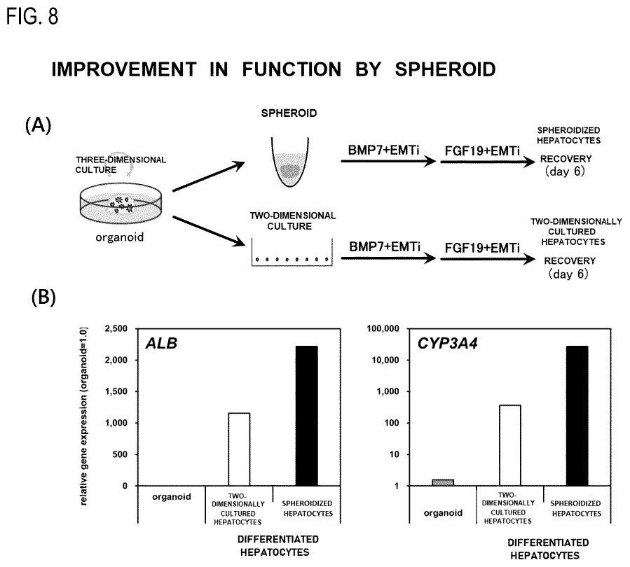

In this Example, spheroidized cells produced from hepatic organoid cells were evaluated for their function. The hepatic organoid cells were seeded at 7.5×10³ cells/well on Nunclon Sphera 96U Bottom Plate (manufactured by Thermo Fisher Scientific) serving as a container for spheroid culture, and were cultured with a HCM™ medium having added thereto the EMTi and BMP7 (50 ng/mL) until the third day of the culture to be differentiated. The medium was replaced with a new one on the third day of the culture, and the cells were subjected to spheroid culture with the HCM™ medium having added thereto the EMTi and FGF19 (100 ng/ml) until the sixth day of the culture. Two-dimensionally cultured hepatocytes were cultured by the method illustrated in FIG. 4D until the sixth day of the culture (FIG. 8A).

The respective gene expression levels of ALB and CYP3A4 were measured by a quantitative RT-PCR method for the respective cells after their culture. A gene expression level when hepatic organoids were cultured as follows was defined as 1: a medium was replaced with a new one on the third day of the culture, and the organoids were cultured with the HCM™ medium having added thereto the EMTi and FGF19 (100 ng/ml) until the sixth day of the culture. As a result, in each of the spheroidized cells and the two-dimensionally cultured hepatocytes, the respective gene expression levels of ALB and CYP3A4 showed increasing tendencies as compared to the hepatic organoids (FIG. 8B).

### (Example 9) Evaluation 1 by Long-term Culture

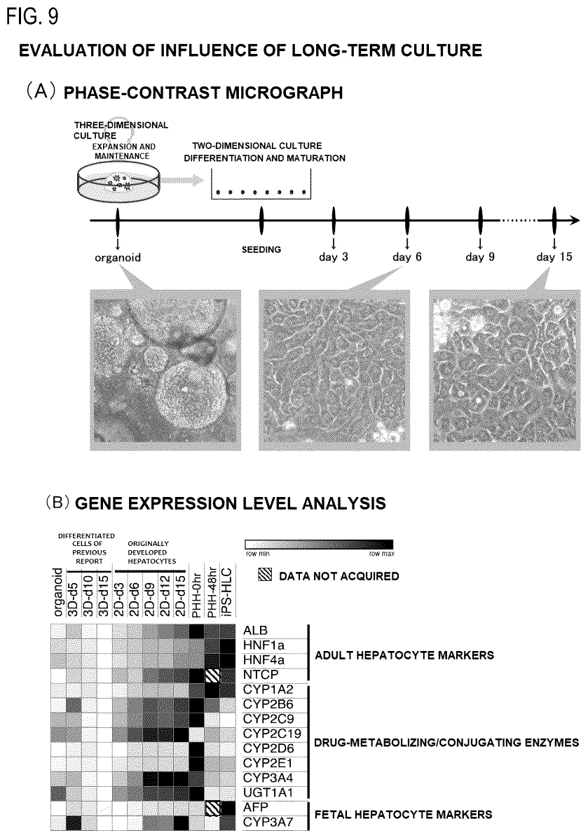

In this Example, the two-dimensional culture of the hepatocytes (2D differentiated cells) of the present invention with a HCM™ medium having added thereto the EMTi, FGF19 (100 ng/ml), and Dex (10 μM) was continued even after the ninth day of the culture. The medium was replaced with a new one every 3 days, and the two-dimensional culture was continued until the 15th day. The morphology of the cells was observed with a phase-contrast microscope. It was suggested that the hepatocytes of the present invention had valvate morphology peculiar to hepatocytes even on the 15th day of the culture, and were hence able to be cultured for a long time period (FIG. 9A).

Various gene expression levels were measured for hepatic organoid cells, the hepatocytes of the related-art method on the 5th day to 15th day of their culture (3D-d5 to d15), the hepatocytes of the present invention on the 5th day to 15th day of their culture (2D-d3 to d15), the human frozen hepatocytes (PHH-0 hr and PHH-48 hr), and human iPS cell-derived hepatocyte-like cells (iPS-HLCs). The results are shown in a heatmap (FIG. 9B). In the hepatocytes of the present invention, the gene expression levels of adult hepatocyte markers (ALB, HNF1a, and HNF4a), and drug-metabolizing enzymes (various CYP enzymes) and conjugating enzymes increased with time until the 3rd day to 15th day of the two-dimensional culture.

### (Example 10) Evaluation 2 by Long-term Culture

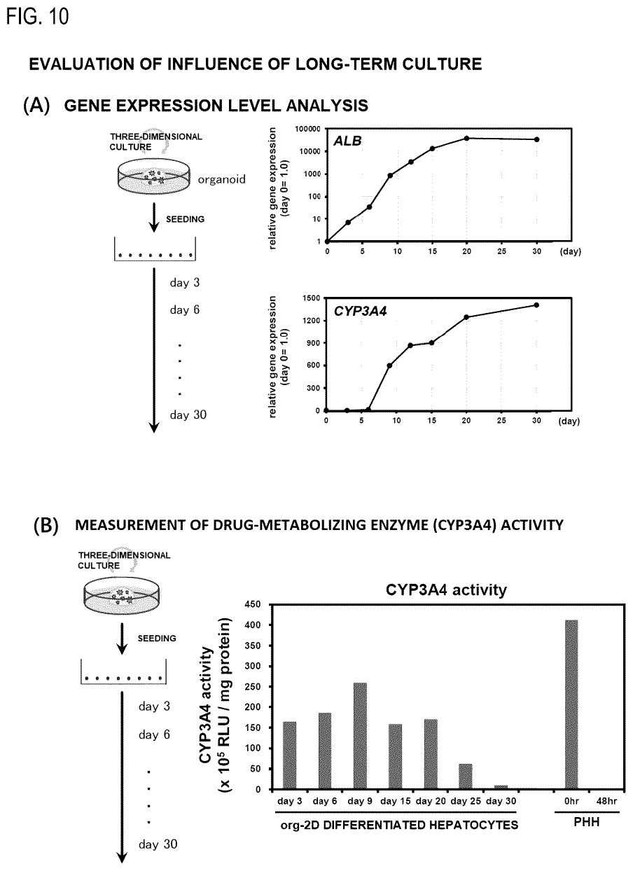

In this Example, the two-dimensional culture of the hepatocytes (2D differentiated cells) of the present invention with a HCM™ medium having added thereto the EMTi, FGF19 (100 ng/ml), and Dex (10 μM) was continued even after the ninth day of the culture. The medium was replaced with a new one every 3 days, and the two-dimensional culture was continued until the 30th day. The respective gene expression levels of ALB and CYP3A4 were measured by a quantitative RT-PCR method for the respective cells. An expression level at the time of the start of the two-dimensional culture (0 day) was defined as 1 (FIG. 10A).

The above-mentioned cells that had been two-dimensionally cultured for 30 days were evaluated for their function by using activity for the drug-metabolizing enzyme CYP3A4 as an indicator. As a comparative example, with regard to primary human hepatocytes (PHHs), frozen cells were thawed, and were then cultured with a HCM™ medium for 48 hours. A culture supernatant immediately after the freezing and thawing was referred to as “PHH-Ohr”, and a culture supernatant after the 48 hours of culture was referred to as “PHH-48 hr”. The CYP3A4 activity was measured by the same approach as that of Example 5. The CYP3A4 activity on and after the ninth day of the culture reduced (FIG. 10B).

### (Example 11) Production of iPS Cell-derived Hepatic Organoid

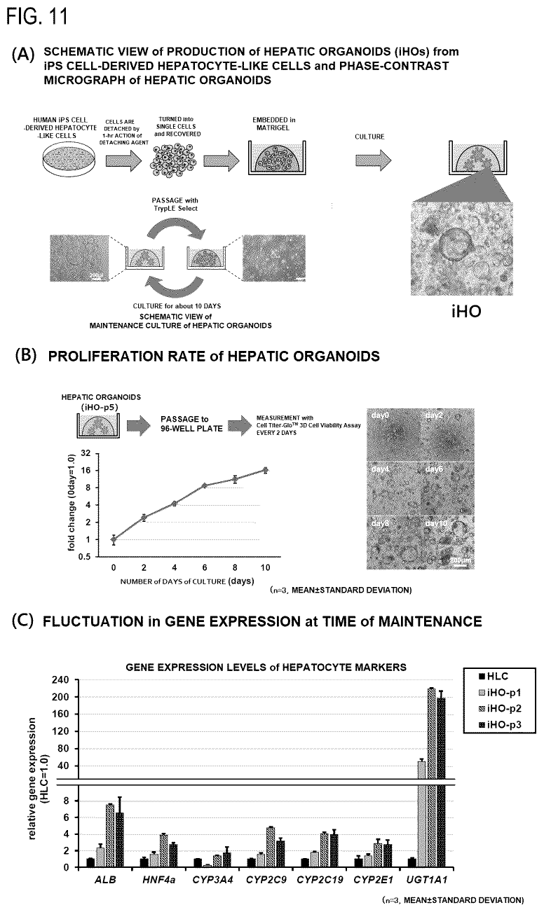

In this Example, hepatic organoids were produced from iPS cell-derived hepatocyte-like cells. TrypLE Select™ was caused to act on the iPS cell-derived hepatocyte-like cells (HLCs) for 1 hour to detach the cells. Single cells were recovered by two-stage strainer treatment (200 μL and 70 μL). The recovered cells were subjected to centrifugation treatment and suspended in PBS+Y27632 (10 μM). The suspension was further subjected to centrifugation treatment, and the recovered cells were embedded in Matrigel™ (estimated value of a seeding density=5×10⁵ cells/40 μL droplet). After the cells had been incubated at 37° C. for 15 minutes, a medium for a liver organoid (a Chol-med medium or a Hep-med medium) was added thereto, and the cells were cultured for 6 days while the medium was replaced with a new one every 2 days. The Chol-med medium was produced with reference to the description of Non Patent Literature 1. In this Example and Examples below, iPS cell-derived hepatic organoids produced with the Chol-med medium are referred to as “iPSC-derived HLC organoids (iHOs),” and such iPS cell-derived hepatic organoids that the Chol-med medium is switched to the Hep-med medium in the middle of iHO production are referred to as “iHOs-Hep”. The cells of each of the produced iHOs and iHOs-Hep were treated with TrypLE Select™ and subcultured every 10 days (FIG. 11A).

A proliferation curve was shown for a zeroth day to a tenth day after seeding in hepatic organoids at passage 5 (iHOs-p5). A cell viability was measured with Cell Titer-Glo™ 3D Cell Viability Assay (Promega) every 2 days, and a proliferation rate on the first day of the culture was shown as 1.0 (FIG. 11B). In addition, the morphology of the cells was observed with a phase-contrast microscope (FIG. 11B).

The gene expression levels of hepatocyte markers were measured for the iPS cell-derived hepatocyte-like cells (HLCs), hepatic organoids at passage 1 (iHOs-p1), hepatic organoids at passage 2 (iHOs-p2), and hepatic organoids at passage 3 (iHOs-p3). When the HLCs were turned into organoids, the gene expression levels of the hepatocyte markers were largely increased as compared to those of the HLCs before being turned into the organoids (FIG. 11C).

### (Example 12) Two-dimensional Culture from iPS Cell-derived Hepatic Organoid

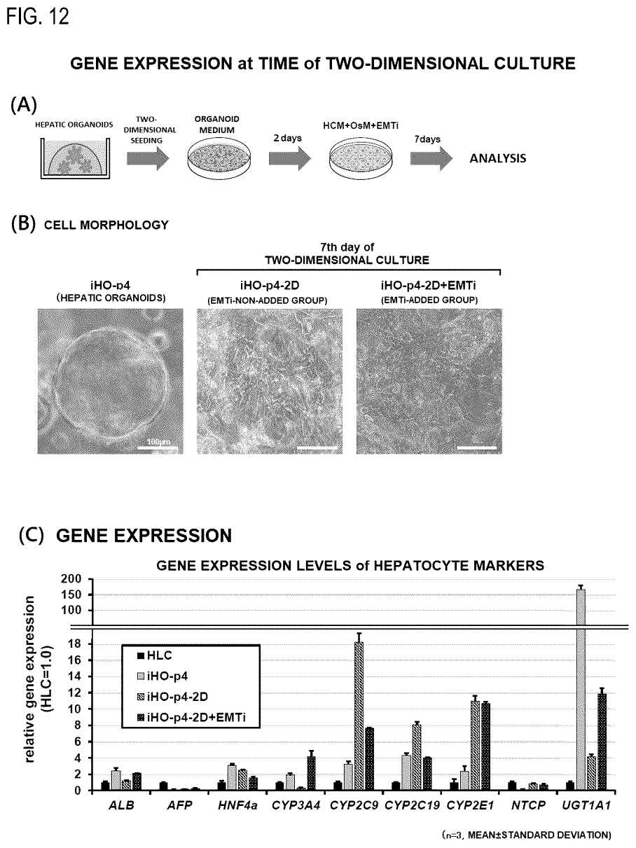

In this Example, the hepatic organoids (iHOs) produced in Example 11 were two-dimensionally cultured, and their hepatic function was identified. Hepatic organoids at passage 4 (iHOs-p4) produced by the method of Example 11 were removed from the Matrigel, and were seeded at 1.2×10⁵ cells/well on a 96-well plate, followed by their two-dimensional culture with an EMTi-containing medium for a hepatic organoid (Chol-med medium) for 2 days. After that, the organoids were two-dimensionally cultured with a HCMTM medium containing the EMTi and 20 ng/mL oncostatin M (OsM) for 7 days (FIG. 12A). The cell morphologies of the hepatic organoids at passage 4 (iHOs-p4) and the two-dimensionally cultured iHOs-p4 were identified. With regard to the two-dimensionally cultured iHOs-p4, the cell morphology of a system cultured with an EMTi-free HCM™ medium was also identified. The morphology of the two-dimensionally cultured iHOs-p4 was close to that of hepatocytes (FIG. 12B).

The gene expression levels of hepatocyte markers were analyzed by a quantitative RT-PCR method for the iPS cell-derived hepatocyte-like cells (HLCs), the hepatic organoids at passage 4 (iHOs-p4), and the two-dimensionally cultured iHOs-p4. With regard to the two-dimensionally cultured iHOs-p4, the gene expression levels of the system cultured with the EMTi-free medium were also identified. Data on the respective gene expression levels was obtained while a value for the HLC was defined as 1.0. The two-dimensional culture tended to increase the expression of each CYP gene. In contrast, a fetal hepatocyte marker AFP maintained expression lower than that of the HLCs, and the expression of a gene except UGT1A1 was also higher than or comparable to the gene expression of the HLCs. The foregoing suggested maturation into hepatocytes by the two-dimensional culture (FIG. 12C).

### (Example 13) Influence of Cryopreservation on iPS Cell-derived Hepatic Organoid (iHO)

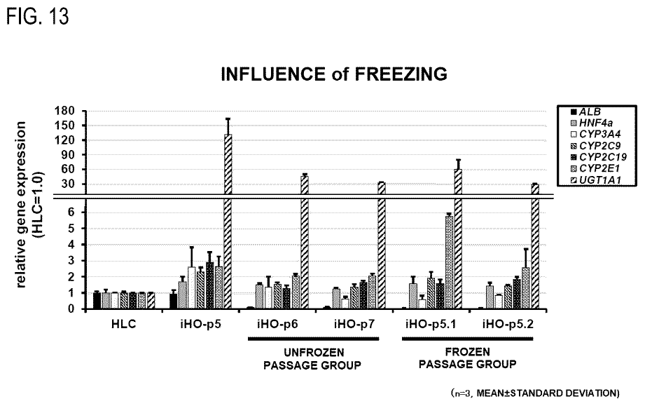

In this Example, the gene expression levels of hepatocyte markers were analyzed by a quantitative RT-PCR method for the iPS cell-derived hepatocyte-like cells (HLCs), the hepatic organoids at passage 5 (iHOs-p5) produced by the method of Example 11, and an unfrozen passage group and a frozen passage group thereof. The gene expression levels of hepatic organoids at passage 6 (iHOs-p6) and hepatic organoids at passage 7 (iHOs-p7) serving as the unfrozen passage group, and those of hepatic organoids obtained as follows, the hepatic organoids serving as the frozen passage group, were identified: the iHOs-p5 were cryopreserved, and were then reseeded to provide hepatic organoids at passage 1 (iHOs-p5.1) and hepatic organoids at passage 2 (iHOs-p5.2). It was revealed that the cells were able to be frozen because the frozen group showed gene expression levels comparable to those of the unfrozen group (FIG. 13).

### (Example 14) Investigation 1 on Conditions for Two-dimensional Culture of iPS Cell-derived Hepatic Organoid (iHO)

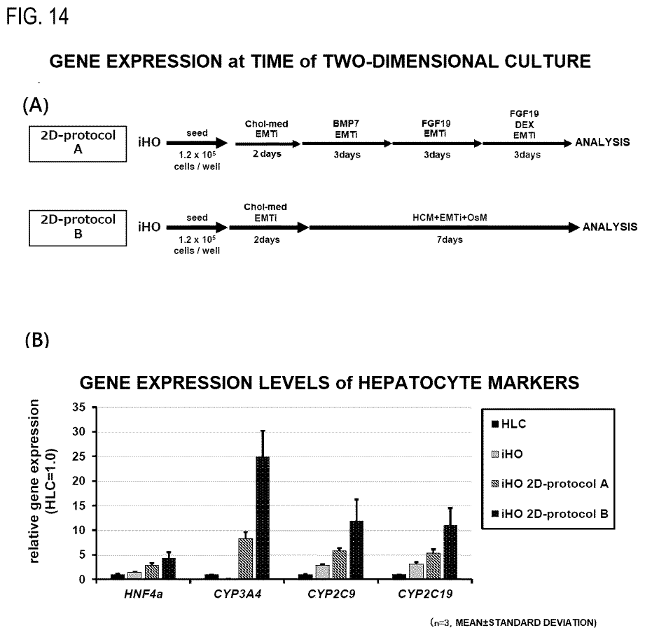

In this Example, conditions for the two-dimensional culture of the hepatic organoids (iHOs) produced in Example 11 were investigated. In a protocol A, hepatic organoid cells obtained by removing the hepatic organoids (iHOs) produced by the method of Example 11 from the Matrigel™ were seeded at 1.2×10⁵ cells/well on a 96-well plate, and were two-dimensionally cultured with an EMTi-containing Chol-med medium for 2 days, followed by their two-dimensional culture with a HCM™ medium containing the EMTi and BMP7 (50 ng/ml) for 3 days. Next, the medium was replaced with a HCM™ medium containing the EMTi and FGF19 (100 ng/mL), and the cells were two-dimensionally cultured therewith for 3 days. Further, the cells were two-dimensionally cultured with a HCM™ medium containing the EMTi, FGF19 (100 ng/ml), and Dex (10 μM) for 3 days (FIG. 14A). In a protocol B, the hepatic organoid cells were seeded at 1.2×10⁵ cells/well on a 96-well plate, and were two-dimensionally cultured with the EMTi-containing Chol-med medium for 2 days. Next, the medium was replaced with a HCM™ medium containing the EMTi and OsM (20 ng/mL), and the cells were two-dimensionally cultured therewith for 7 days (FIG. 14A). In each of the protocols A and B, the two-dimensional culture increased the gene expression levels of hepatocyte markers. In particular, the gene expression level of CYP3A4 important for drug metabolism significantly increased. It was suggested from the foregoing that the two-dimensional culture improved the function of an iHO-derived cultured hepatocyte.

### (Example 15) Investigation 2 on Conditions for Two-dimensional Culture of iPS Cell-derived Hepatic Organoid

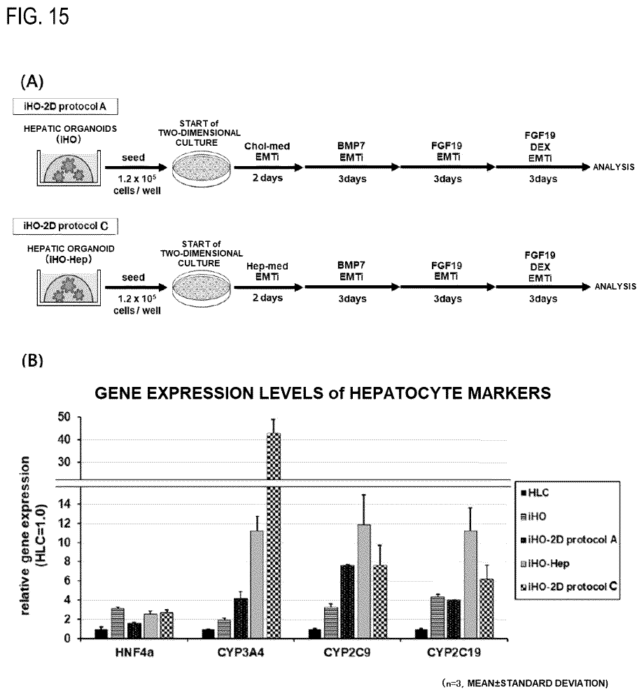

In this Example, conditions for the two-dimensional culture of iPS cell-derived hepatic organoids were investigated. In a protocol A, hepatic organoid cells obtained by removing the hepatic organoids (iHOs) produced by the method of Example 11 from the Matrigel™ were seeded at 1.2×10⁵ cells/well on a 96-well plate, and were two-dimensionally cultured with an EMTi-containing Chol-med medium for 2 days. Next, the cells were two-dimensionally cultured with a HCM™ medium containing the EMTi and BMP7 (50 ng/mL) for 3 days. Next, the medium was replaced with a HCM™ medium containing the EMTi and FGF19 (100 ng/mL), and the cells were two-dimensionally cultured therewith for 3 days. Further, the cells were two-dimensionally cultured with a HCM™ medium containing the EMTi, FGF19 (100 ng/ml), and Dex (10 μM) for 3 days (FIG. 15A). In a protocol C, iHO-Hep-derived hepatic organoid cells were two-dimensionally cultured in the same manner as in the protocol A except that the cells were two-dimensionally cultured from such iPS cell-derived hepatic organoids (iHOs-Hep) that the Chol-med medium was switched to a Hep-med medium in the middle of iHO production with an EMTi-containing Hep-med medium for 2 days (FIG. 15A).

In each of the methods of the protocols A and C, the two-dimensional culture increased the gene expression levels of hepatocyte markers. In particular, the gene expression level of CYP3A4 important for drug metabolism significantly increased. It was suggested from the foregoing that the two-dimensional culture improved the function of each of iHO-derived and iHO-Hep-derived cultured hepatocytes (FIG. 15B).

### (Example 16) Investigation 3 on Conditions for Two-dimensional Culture of iPS Cell-derived Hepatic Organoid

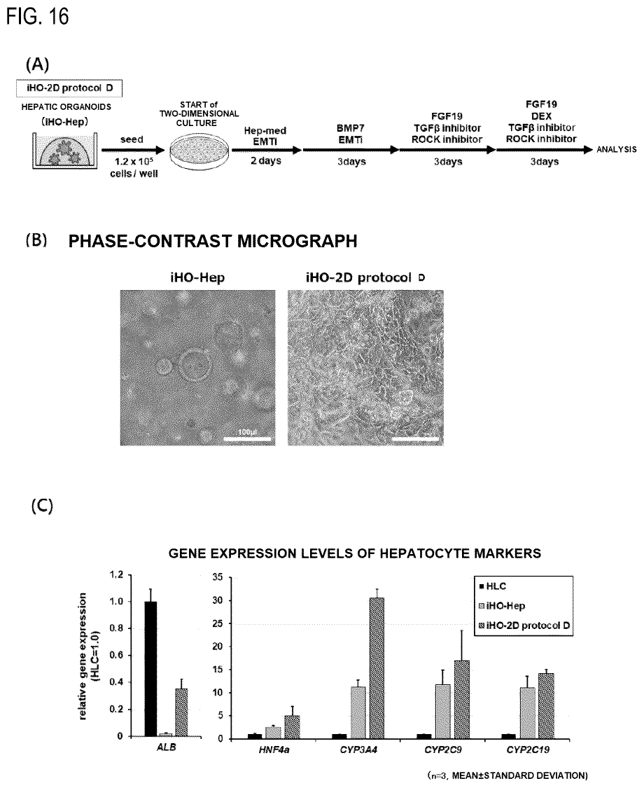

In this Example, as a protocol D, iHO-Hep-derived hepatic organoid cells were two-dimensionally cultured by the same approach as that of the protocol C of Example 15 except that in a culturing step, Y-27632 (ROCK inhibitor, 10 μM) and SB-431542 (TGF-β inhibitor, 2 μM) were used instead of the EMTi (Y-27632 (10 μM), PD0325901 (0.5 μM), and SB-431542 (2 μM)) (FIG. 16A).

As a result of observation with a phase-contrast microscope, valvate cell morphology peculiar to hepatocytes was observed (FIG. 16B). The two-dimensional culture by the method of the protocol D increased the gene expression levels of hepatocyte markers. In particular, the gene expression level of CYP3A4 important for drug metabolism significantly increased. It was suggested from the foregoing that when the two-dimensional culture was performed with a medium, which was free of the MEK inhibitor, and contained the ROCK inhibitor and the TGF-β inhibitor, instead of the EMTi containing the ROCK inhibitor, the MEK inhibitor, and the TGF-β inhibitor, the function of an iHO-Hep-derived cultured hepatocyte was improved (FIG. 16C).

### (Example 17) Investigation 1 on Conditions for Production of iPS Cell-derived Hepatic Organoid

In this Example, the function of a hepatic organoid to be established was identified for the production of a hepatic organoid (iHO) from an iPS cell by a difference in hepatic differentiation induction stage of the cell to be used.

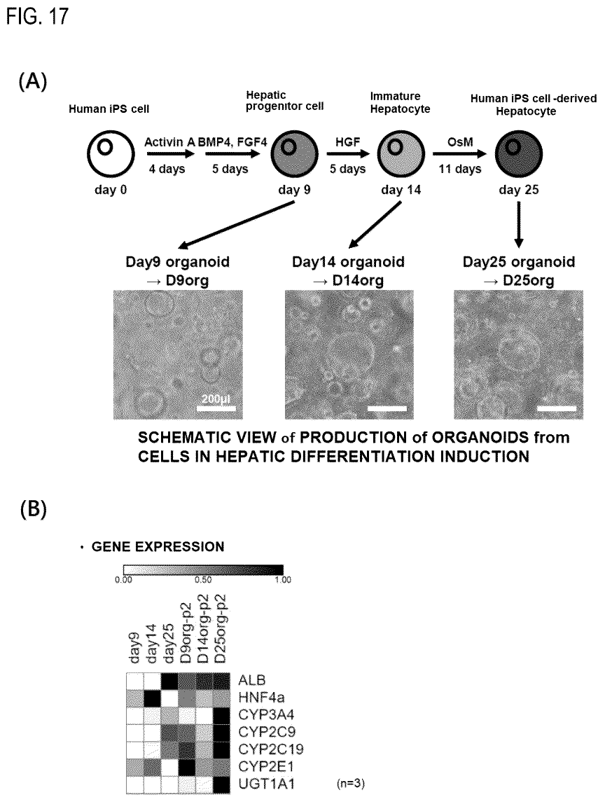

TrypLE Select™ was caused to act on cells at each stage (day 9, day 14, or day 25) of a hepatic differentiation induction process from the iPS cells illustrated in FIG. 17A, and single cells were recovered, subjected to centrifugation treatment, and suspended in PBS+Y27632 (10 μM). The cells were centrifuged and embedded in Matrigel™ (estimated value of a seeding density=1×10⁵ cells/40 μL droplet). After the cells had been incubated at 37° C. for 15 minutes, a medium for their culture was replaced with an EMTi-containing Chol-med medium every 2 days. The cell morphologies of the hepatic organoids (D9orgs, D14orgs, and D25orgs) 9 days, 14 days, and 25 days after the culture were observed with a phase-contrast microscope (FIG. 17A).

The respective gene expression levels of hepatocyte markers in each of the organoids at passage 2 (D9orgs-p2, D14orgs-p2, and D25orgs-p2) produced from the cells at the respective stages of the hepatic differentiation induction process from the iPS cells were measured by a quantitative RT-PCR method, and their heatmap was produced with MORPHEUS. In the organoids produced from each of the differentiation induction stages, the respective gene expression levels of the hepatocyte markers increased. Comparison between the organoids showed that the gene expression levels tended to be highest in the D25orgs (FIG. 17B).

### (Example 18) Investigation 2 on Conditions for Production of iPS Cell-derived Hepatic Organoid

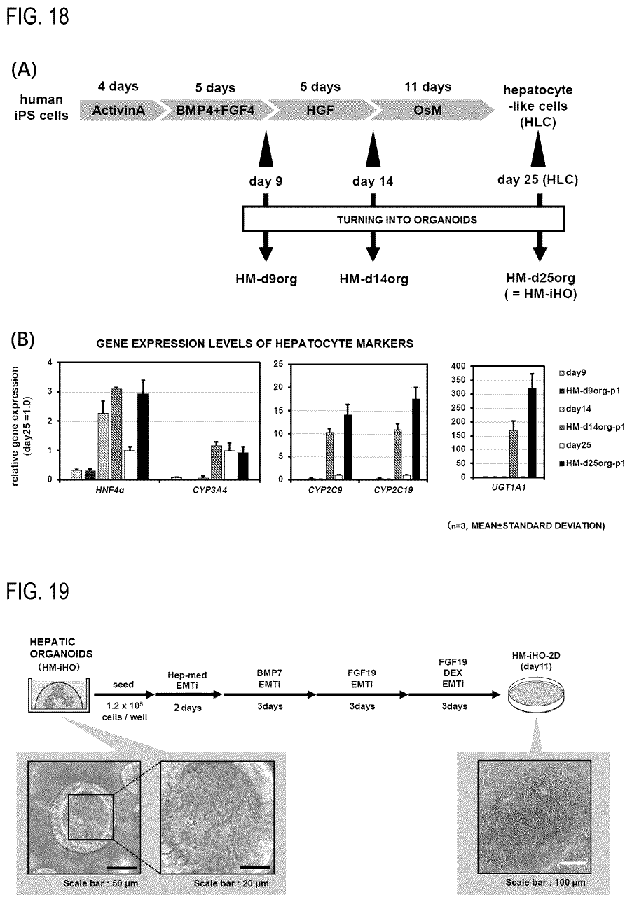

In this Example, the function of a hepatic organoid to be established was identified for an iPS cell-derived hepatic organoid produced with a Hep-med medium by a difference in hepatic differentiation induction stage to be used. Hepatic organoids were produced by the same approach as that of Example 17 except that the Hep-med medium was used instead of the Chol-med medium. Specifically, the iPS cell-derived hepatic organoids (HM-d9orgs, HM-d14orgs, and HM-d25orgs) were produced from cells at the respective stages (day 9, day 14, and day 25) of a hepatic differentiation induction process 9 days, 14 days, and 25 days after the culture of iPS cells illustrated in FIG. 18A. In this Example and Examples below, hepatic organoids produced with the Hep-med medium were used. In addition, in this Example and Examples below, cells on day 25 of the hepatic differentiation induction process refer to human iPS cell-derived hepatocyte-like cells (HLCs), and iPS cell-derived hepatic organoids (HM-d25orgs) produced from the cells on day 25 of the hepatic differentiation induction process with the Hep-med medium are referred to as “HM-iHOs”.

The gene expression levels of hepatocyte markers in the cells at the respective stages (day 9, day 14, and day 25) of the hepatic differentiation induction process and the respective hepatic organoids at passage 1 (HM-d9orgs-p1, HM-d14orgs-p1, and HM-d25orgs-p1) produced from the cells at the respective stages of the hepatic differentiation induction process from the iPS cells were analyzed by a qRT-PCR method. Data on the respective gene expression levels was obtained while a value for the cells on day 25 was defined as 1.0. Gene expression tended to be highest in the organoids produced from the cells on day 25 of the differentiation induction (FIG. 18B).

### (Example 19) Two-dimensional Culture from iPS Cell-derived Hepatic Organoid (HM-iHO)

In this Example, HM-iHO-derived hepatic organoid cells were two-dimensionally cultured from the hepatic organoids (HM-iHOs) produced in Example 18 in the same manner as in the protocol C of Example 15 (FIG. 19). The hepatic organoids (HM-iHOs) produced with the Hep-med medium and a two-dimensional culture group (HM-iHO-2D, day 11) obtained by two-dimensionally culturing the HM-iHOs for 11 days were observed with a phase-contrast microscope (FIG. 19). The hepatic organoids and the two-dimensional culture group showed hepatocyte-like polygonal cell morphologies. The term “EMTi” refers to the combination of 0.5 μM PD0325901, 2 μM SB-431542, and 10 μM Y-27632.

### (Example 20) Hepatocyte Function Evaluation (Gene Expression Level of Hepatocyte Marker)

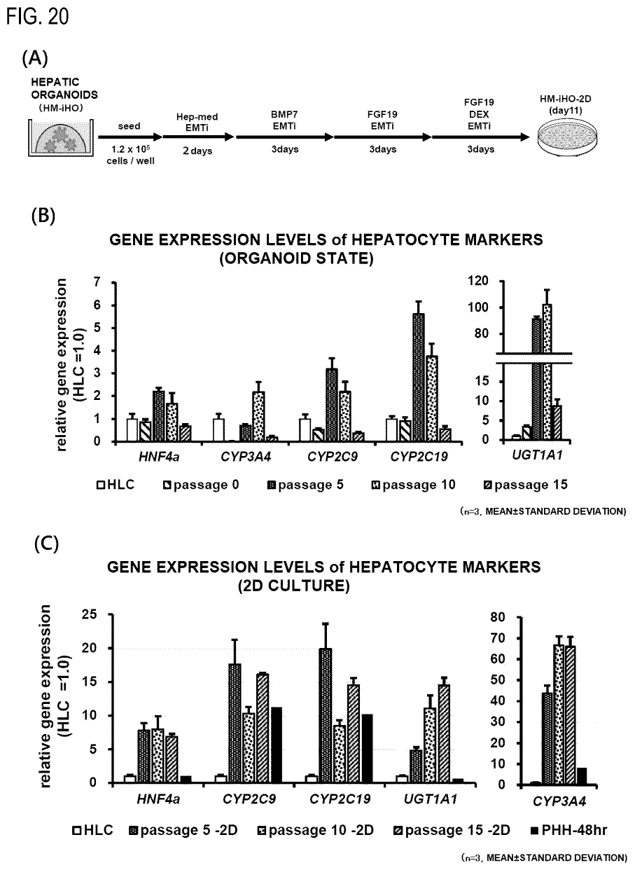

In this Example, HM-iHO-derived hepatic organoid cells were two-dimensionally cultured from the hepatic organoids (HM-iHOs) produced in Example 18 by the same approach as that of Example 19 (FIG. 20A). The gene expression levels of hepatocyte markers in the hepatic organoids (HM-iHOs) at passage 0, passage 5, passage 10, and passage 15 produced with the Hep-med medium were analyzed by a qRT-PCR method (FIG. 20B). Data on the respective gene expression levels was obtained while a value for HLCs was defined as 1.0. The gene expression levels of the hepatocyte markers in products (passage 5-2D, passage 10-2D, and passage 15-2D) obtained by two-dimensionally culturing the hepatic organoids (HM-iHOs) at passage 5, passage 10, and passage 15, which had been produced with the Hep-med medium, for 11 days were analyzed by the qRT-PCR method (FIG. 20C). The term “PHH-48 hr” refers to a product obtained by culturing primary human hepatocytes for 48 hours. Data on the respective gene expression levels was obtained while a value for the HLCs was defined as 1.0. In an organoid state, a large difference in gene expression was found between the numbers of passages. However, the two-dimensional culture reduced the difference to stabilize the gene expression levels.

### (Example 21) Evaluation 3 by Long-term Culture

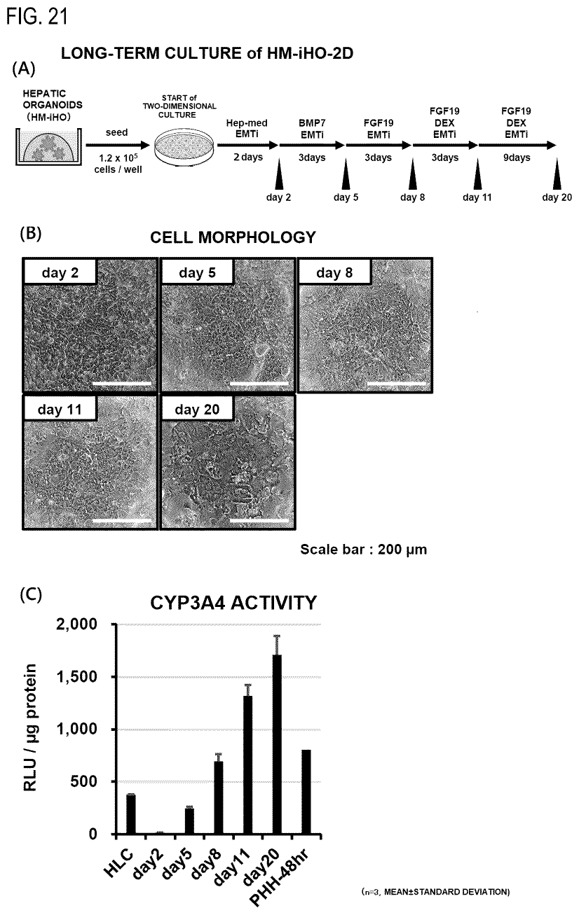

In this Example, the hepatic organoids (HM-iHOs) produced in Example 18 were two-dimensionally cultured (FIG. 21A). The organoids were two-dimensionally cultured by the same approach as that of Example 19, and their two-dimensional culture with a HCM™ medium having added thereto the EMTi, FGF19 (100 ng/mL), and Dex (10 μM) was continued even after the 11th day of the two-dimensional culture. After the 11th day of the two-dimensional culture, the medium was replaced with a new one every 9 days, and the two-dimensional culture was continued until the 20th day. Cell morphologies on day 2, day 5, day 8, day 11, or day 20 of the two-dimensional culture were observed with a phase-contrast microscope (FIG. 21B), and CYP3A4 activity was analyzed (FIG. 21C). The term “PHH-48 hr” refers to a product obtained by culturing primary human hepatocytes for 48 hours. It was suggested that the HM-iHOs were able to be two-dimensionally cultured for about 3 weeks, and hence were able to be cultured for a long time period that was unprecedentedly long.

### (Example 22) Hepatocyte Function Evaluation (Bile Canaliculus Formation)

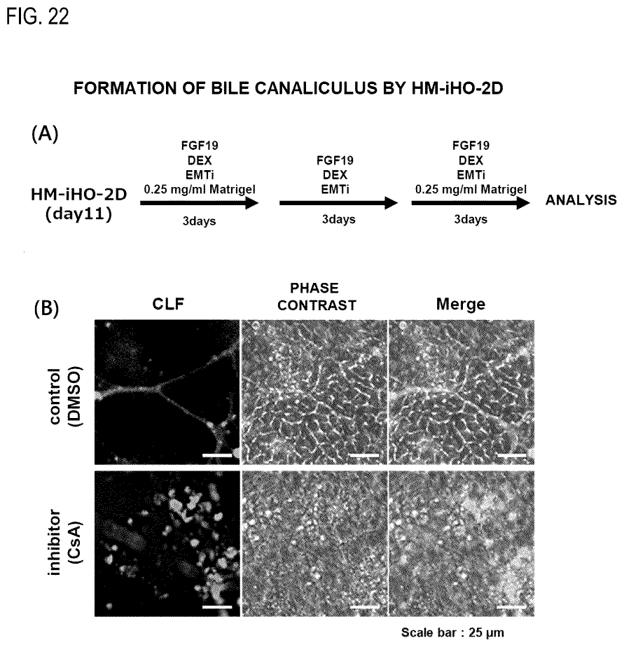

In this Example, cells were two-dimensionally cultured from the hepatic organoids (HM-iHOs) produced in Example 18 by the same approach as that of Example 19. Next, the medium was replaced with a HCM™ medium containing the EMTi, FGF19 (100 ng/ml), Dex (10 μM), and Matrigel™ (0.25 mg/mL), and the cells were cultured therewith for 3 days. Next, the medium was replaced with a HCM™ medium containing the EMTi, FGF19 (100 ng/mL), and Dex (10 μM), and the cells were cultured therewith for 3 days. Next, the medium was replaced with the HCM™ medium containing the EMTi, FGF19 (100 ng/ml), Dex (10 μM), and the Matrigel™ (0.25 mg/mL), and the cells were cultured therewith for 3 days. Thus, sandwich culture was performed (FIG. 22A). A bile canaliculus-forming ability on the 20th day of the two-dimensional culture was analyzed (FIG. 22B). In a control group on which DMSO had been caused to act, the discharge of fluorescein-labeled bile acid (cholyl-lysyl-fluorescein: CLF) into a bile canaliculus was able to be observed. In contrast, in an inhibitor group, the discharge was inhibited by cyclosporin A (CsA).

### (Example 23) Influence of Cryopreservation on iPS Cell-derived Hepatic Organoid (HM-iHO)

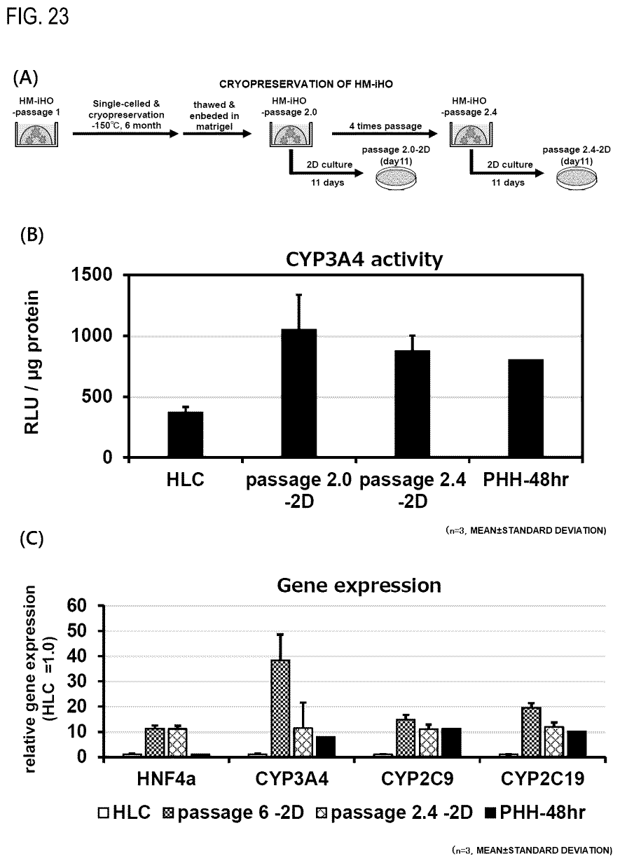

In this Example, the hepatic organoids (HM-iHOs) produced in Example 18 were passaged numbers of times of passage of 2.0 and 2.4, and the organoids at passage 2.0 and passage 2.4 were two-dimensionally cultured by the same approach as that of Example 19 for 11 days, followed by a hepatocyte function evaluation (FIG. 23A). The CYP3A4 activity of a product (passage 2.0-2D or passage 2.4-2D) obtained by two-dimensionally culturing each of the hepatic organoids for 11 days was analyzed (FIG. 23B). The term “passage 2.0” refers to hepatic organoids obtained by cryopreserving the hepatic organoids (HM-iHOs) produced in Example 18, and then reseeding the organoids, and the term “passage 2.4” refers to hepatic organoids at passage 4 obtained by cryopreserving the hepatic organoids (HM-iHOs) produced in Example 18, and then reseeding the organoids. The term “PHH-48 hr” refers to a product obtained by culturing primary human hepatocytes for 48 hours.

Further, the gene expression levels of hepatocyte markers in products (passage 6-2D and passage 2.4-2D) obtained by two-dimensionally culturing hepatic organoids at passage 6, which served as an unfrozen group of the hepatic organoids (HM-iHOs) produced in Example 18, and hepatic organoids at passage 2.4, which served as a frozen group thereof, for 11 days were analyzed by a qRT-PCR method (FIG. 23C). The term “PHH-48 hr” refers to a product obtained by culturing primary human hepatocytes for 48 hours. The HM-iHOs were able to be cryopreserved because the HM-iHOs after the cryopreservation showed high CYP3A4 activity and high gene expression when two-dimensionally cultured.

### (Example 24) Comparison 1 between iPS Cell-derived Hepatic Organoids

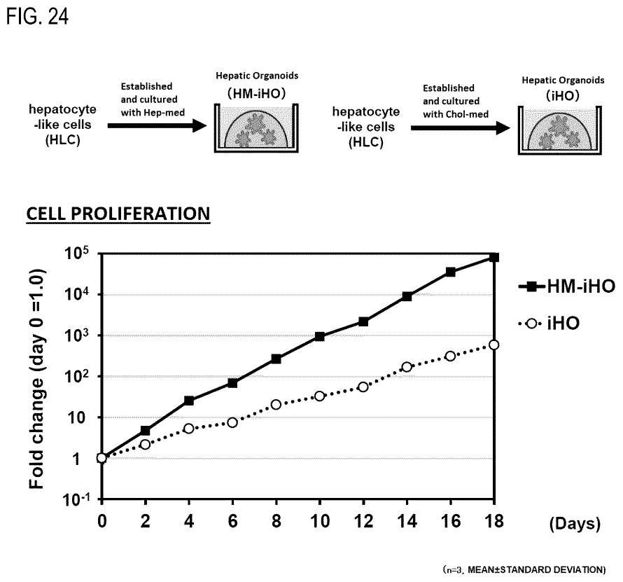

In this Example, the cell proliferation potency of each of the hepatic organoids (HM-iHOs) produced in Example 18 and the hepatic organoids (iHOs) produced in Example 11 was analyzed. The HM-iHOs were superior in cell proliferation potency to the iHOs (FIG. 24).

### (Example 25) Comparison 2 between iPS Cell-derived Hepatic Organoids

In this Example, the hepatocyte functions of the hepatic organoids (HM-iHOs) produced in Example 18 and the hepatic organoids (iHOs) produced in Example 11 were compared to each other.

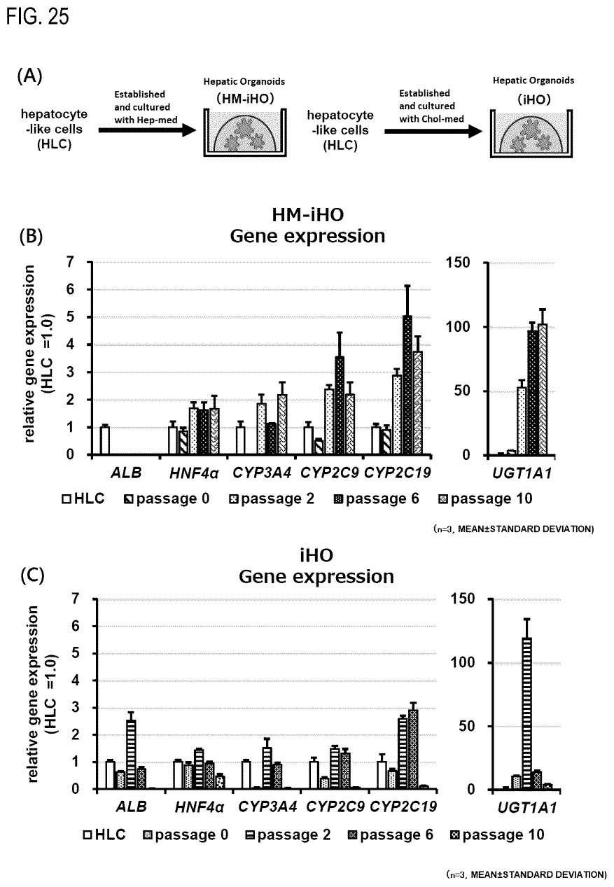

Hepatic organoids were produced by the approach of Example 18 or Example 11 (FIG. 25A). The gene expression levels of hepatocyte markers in the hepatic organoids (HM-iHOs) produced in Example 18, and the hepatic organoids (iHOs) produced in Example 11, at passages 0, 2, 6, and 10 were analyzed by a qRT-PCR method (FIG. 25B and FIG. 25C). Data on the respective gene expression levels was obtained while a value for HLCs was defined as 1.0. The HM-iHOs were superior in gene expression during maintenance culture to the iHOs.

### (Example 26) Comparison 3 between iPS Cell-derived Hepatic Organoids

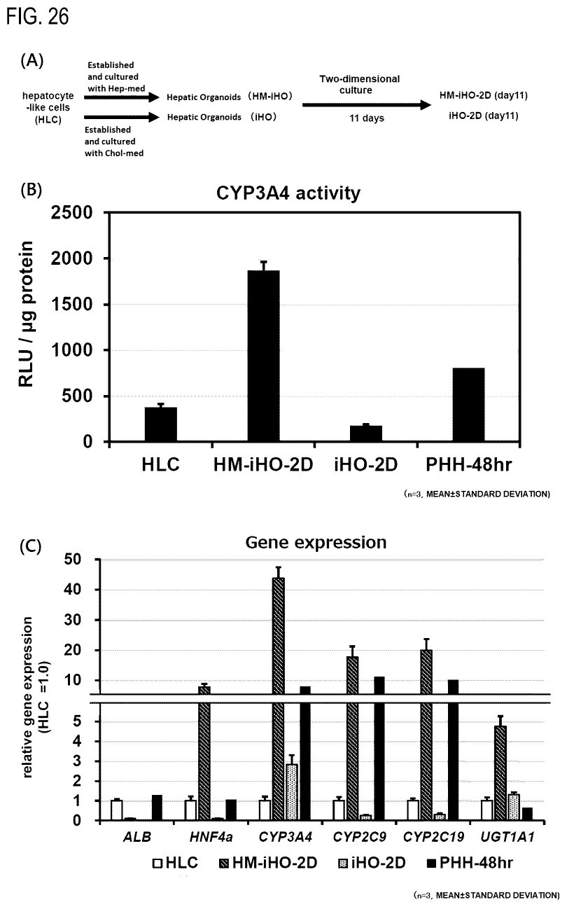

In this Example, the hepatic organoids (HM-iHOs) produced in Example 18 were two-dimensionally cultured by the same approach as that of Example 19 for 11 days, and the hepatic organoids (iHOs) produced in Example 11 were two-dimensionally cultured by the same approach as that of the protocol A of Example 15 for 11 days (FIG. 26A). The hepatocyte functions of products (HM-iHOs-2D and iHOs-2D) obtained by two-dimensionally culturing the hepatic organoids (HM-iHOs) produced in Example 18 and the hepatic organoids (iHOs) produced in Example 11 for 11 days were compared to each other.

The CYP3A4 activity of each of the HM-iHOs-2D and the iHOs-2D was analyzed (FIG. 26B). The term “PHH-48 hr” refers to a product obtained by culturing primary human hepatocytes for 48 hours. Further, the gene expression levels of hepatocyte markers in the HM-iHOs-2D and the iHOs-2D were analyzed by a qRT-PCR method (FIG. 26C). The term “PHH-48 hr” refers to a product obtained by culturing primary human hepatocytes for 48 hours. The HM-iHOs were superior in hepatic function after the two-dimensional culture to the iHOs.

### (Example 27) Two-dimensional Culture of Frozen Organoid

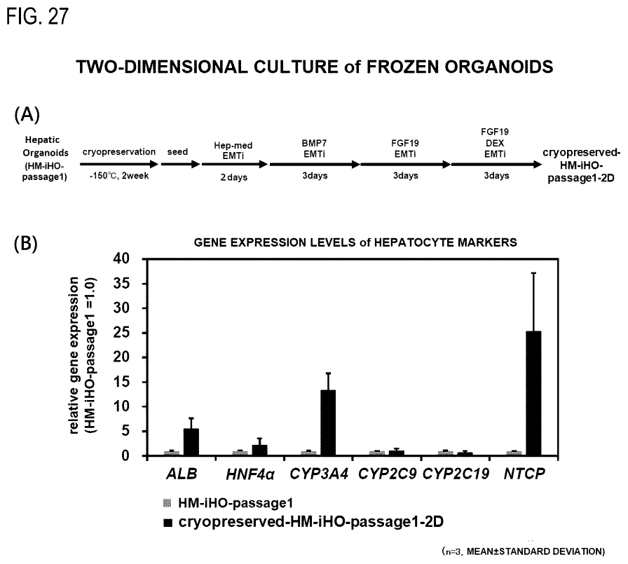

In this Example, the hepatic organoids (HM-iHOs) at passage 1 (HM-iHOs-passage 1) produced in Example 18 were cryopreserved at −150° C. for 2 weeks. After that, the organoids were seeded, and were two-dimensionally cultured by the same approach as that of Example 19 for 11 days, followed by a hepatocyte function evaluation (FIG. 27A). Specifically, TrypLE Select was caused to act on the hepatic organoids (HM-iHOs) at passage 1 (HM-iHOs-passage 1) produced in Example 18 to provide single cells, and the cells were recovered. The recovered cells were suspended in STEM-CELLBANKER, loaded into cryopreservation equipment (Bicell), and left at rest at −80° C. overnight. After that, the cells were transferred to an environment at −150° C. and left at rest for 2 weeks. The cells were incubated in a hot water bath at 37° C. for 90 seconds to be defrosted, and were suspended in PBS, followed by centrifugation to recovery the cells. The cells after their cell count were two-dimensionally cultured at the same cell density as that, and under the same culture conditions as those, of Example 19 for 11 days to provide cryopreserved-HM-iHOs-passage 1-2D.

The gene expression levels of hepatocyte markers were analyzed by a qRT-PCR method for the hepatic organoids (HM-iHOs) at passage 1 (HM-iHOs-passage 1) before their freezing and the organoids (cryopreserved-HM-iHOs-passage 1-2D), which were obtained by cryopreserving the hepatic organoids at passage 1, and then seeding and two-dimensionally culturing the organoids (FIG. 27B). Data on the respective gene expression levels was obtained while a value for the HM-iHOs-passage 1 was defined as 1.0. Even when the hepatic organoids (HM-iHOs) were frozen, and were two-dimensionally cultured in a direct manner, the organoids held a sufficient hepatocyte function.

### INDUSTRIAL APPLICABILITY

As described above, in the cultured hepatocyte of the present invention, the gene expression level of each of the drug-metabolizing enzymes is increased as compared to those of the hepatic organoid. Further, the cultured hepatocyte of the present invention maintains not only the gene expression of the drug-metabolizing enzyme but also high activity for the drug-metabolizing enzyme. Accordingly, the cultured hepatocyte can be effectively utilized in an in vitro pharmacokinetics evaluation. Further, the cultured hepatocyte of the present invention shows excellent sensitivity to a hepatopathy-expressing drug. According to the method of producing a cultured hepatocyte of the present invention, a large amount of cells each having such excellent performance can be supplied in one and the same lot, and hence hepatocytes each having certain quality can be supplied semipermanently. Further, the pluripotent stem cell-derived hepatic organoid of the present invention tends to highly express the gene of a drug-metabolizing enzyme, and can be cryopreserved. The provision of such excellent hepatic organoid enables the production of an excellent hepatocyte or the like.

Such hepatocytes each having excellent quality or a cell population thereof may be used in a kit for evaluating pharmacokinetics or evaluating drug toxicity. Although it has heretofore been difficult to perform an in vitro pharmacokinetics evaluation or a drug toxicity test in a hepatocyte at a certain level, a large-scale and high-accuracy safety evaluation system can be provided. Thus, the hepatopathy risk of a compound can be predicted at an early stage in the research and development of a medicine such as drug discovery and development, and the prediction is extremely useful in increasing the success rate of the research and development, and curtailing costs therefor and the period thereof.

Further, the cultured hepatocyte of the present invention may be used in regenerative medicine or cell therapy in, for example, a case in which liver transplantation has heretofore been required. The regenerative medicine based on the cultured hepatocyte of the present invention may be capable of repairing a damaged hepatocyte and returning the function of a fibrillated liver to a normal one for a patient having a disease, such as hepatitis, a fatty liver, autoimmune hepatitis, or a liver cancer, and hence an effective therapeutic effect can be expected. In addition, the regenerative medicine using the cultured hepatocyte of the present invention is extremely excellent because of the following reason: according to the medicine, a cultured hepatocyte having higher immune compatibility for a patient can be selected, and the risk of unpreferred viral infection or the like along with organ transplantation can be reduced.

1. A cultured hepatocyte, comprising a hepatic organoid-derived cell, wherein a gene expression level of a drug-metabolizing enzyme of the cultured hepatocyte is increased as compared to a gene expression level of the drug-metabolizing enzyme of a hepatic organoid.

2. The cultured hepatocyte according to claim 1, wherein the drug-metabolizing enzyme is one or a plurality of drug-metabolizing enzymes selected from CYP3A4, CYP1A2, CYP2B6, CYP2C9, CYP2C19, CYP2D6, CYP2E1, and UGT1A1.

3. The cultured hepatocyte according to claim 1, wherein a gene expression level of an adult hepatocyte marker in the cultured hepatocyte is comparable to or more than a gene expression level of the adult hepatocyte marker of the hepatic organoid.

4. The cultured hepatocyte according to claim 3, wherein the adult hepatocyte marker is one or a plurality of markers selected from albumin (ALB), hepatocyte nuclear factor 1-alpha (HNF1a), hepatocyte nuclear factor 1-alpha (HNF4a), and Na⁺-taurocholate co-transporting polypeptide (NTCP).

5. The cultured hepatocyte according to claim 1, wherein the cultured hepatocyte is a two-dimensionally cultured hepatocyte obtained by two-dimensional culture or a spheroidized hepatocyte obtained by spheroid culture.

6. The cultured hepatocyte according to claim 1, wherein the hepatic organoid is a liver cell-derived hepatic organoid or a pluripotent stem cell-derived hepatic organoid.

7. The cultured hepatocyte according to claim 6, wherein the pluripotent stem cell-derived hepatic organoid is an iPS cell-derived hepatic organoid.

8. A method of producing a cultured hepatocyte including a hepatic organoid- derived cell, the method comprising the following steps:
(1) a step of separating a hepatic organoid into a single cell; and
(2) a step of culturing a hepatic organoid cell separated as the single cell, the hepatic organoid cell being seeded on a substrate for two-dimensional culture, to produce a monolayer film.

9. A method of producing a cultured hepatocyte including a hepatic organoid- derived cell, the method comprising the following steps:
(1) a step of separating a hepatic organoid into a single cell; and
(2) a step of culturing a hepatic organoid cell separated as the single cell in an incubator for spheroid formation.

10. The method of producing a cultured hepatocyte according to claim 8, wherein in (2) the culturing step, the culturing is performed by using, as a culture solution, a medium containing one or two or more kinds of humoral factors selected from EGF, OsM, HGF, Dex, BMP4, BMP7, FGF7, FGF10, and FGF19.

11. The method of producing a cultured hepatocyte according to claim 8, wherein in (2) the culturing step, the culturing is performed by using, as a culture solution, a medium containing one or a plurality of kinds of inhibitors selected from a ROCK inhibitor, a TGF-β inhibitor, a MEK inhibitor, and a GSK-3 inhibitor.

12. The method of producing a cultured hepatocyte according to claim 8, wherein in (2) the culturing step, the culturing is performed by using, as a culture solution, a medium containing a MEK inhibitor, a TGF-β inhibitor, and a ROCK inhibitor.

13. The method of producing a cultured hepatocyte according to claim 8, wherein the hepatic organoid is a liver cell-derived hepatic organoid or a pluripotent stem cell-derived hepatic organoid.

14. The method of producing a cultured hepatocyte according to claim 13, wherein the hepatic organoid is the pluripotent stem cell-derived hepatic organoid, and is a hepatic organoid produced from a cell obtained by culturing a pluripotent stem cell for at least 14 days.

15. The method of producing a cultured hepatocyte according to claim 13, wherein the hepatic organoid is the pluripotent stem cell-derived hepatic organoid, and is a hepatic organoid produced from an iPS-derived hepatocyte.

16. The method of producing a cultured hepatocyte according to claim 8, wherein a medium to be used in production and/or culture of the hepatic organoid is a Hep-med medium or a Chol-med medium.

17. A cultured hepatocyte, which is produced by the production method of claim 8.

18. A kit for evaluating pharmacokinetics and/or evaluating drug toxicity, comprising:
the cultured hepatocyte of claim 1; and
a device and/or a reagent required for an inspection.

19. A method of evaluating pharmacokinetics and/or a method of evaluating drug toxicity, comprising using the cultured hepatocyte of claim 1.

20. A medium for culturing a cultured hepatocyte including a hepatic organoid-derived cell, comprising:
1 μM to 50 μM of a ROCK inhibitor; and
0.1 μM to 5 μM of a TGF-β inhibitor.

21. A method of producing a pluripotent stem cell-derived hepatic organoid, comprising a step of producing the pluripotent stem cell-derived hepatic organoid from a cell obtained by culturing a pluripotent stem cell for at least 14 days.

22. A method of producing a pluripotent stem cell-derived hepatic organoid, comprising a step of producing the pluripotent stem cell-derived hepatic organoid from an iPS-derived hepatocyte.

23. A pluripotent stem cell-derived hepatic organoid, which is produced by the production method of claim 21.

24. A pluripotent stem cell-derived hepatic organoid, the pluripotent stem cell-derived hepatic organoid being intended for use in two-dimensional culture or spheroid culture.

25. The pluripotent stem cell-derived organoid according to claim 24, wherein the pluripotent stem cell-derived hepatic organoid is an iPS cell-derived hepatic organoid.
